(19) United States
(12) Patent Application Publication
(10) Pub. No.: US 2026/0237472 A1
(43) Pub. Date: Aug. 13, 2026

(54) DATA PIPELINE FOR HETEROGONOUS CLINICAL DATA INGESTION, NORMALIZATION, ENRICHMENT AND MANAGEMENT

(71) Applicant: GE Precision Healthcare LLC, Waukesha, WI (US)

(72) Inventors: Jin Wang, Seattle, WA (US); Gerasimos Arvanitis, Kenmore, WA (US); Abderrazek Boufahja, Illkirch-Graffenstaden (FR); Varsha Meghana Kanmuri, Philadelphia, PA (US); Andreas Tzanetakis, Madison, WI (US); Tanmay Verma, Sammamish, WA (US); Abu Mirza, Redmond, WA (US)

(21) Appl. No.: 19/049,121

(22) Filed: Feb. 10, 2025

(51) Int. Cl. G16H 10/60; G16H 30/20

(52) U.S. Cl. CPC G16H 10/60; G16H 30/20

(57) ABSTRACT

A data pipeline for heterogonous clinical data ingestion, normalization, enrichment and management is described. In an example, a method can comprise receiving, by a system comprising a processor, a bundle of healthcare resources configured in accordance with a Fast Healthcare Interoperability Resources (FHIR) standard. The method further comprises tracking, by the system, information regarding existing healthcare resources stored in a FHIR datastore in a tracking database, wherein the information comprises logical identifiers for the existing healthcare resources associated with the existing healthcare resources in the FHIR datastore and healthcare identifiers for the existing healthcare resources, and processing, by the system, the bundle using a data refinement process and the tracking database, wherein the data refinement process comprises updating the tracking database to comprise new information extracted from the bundle.

### TECHNICAL FIELD

This disclosure relates generally to a data pipeline for heterogonous clinical data ingestion, normalization, enrichment and management.

### BACKGROUND

Interoperability of clinical data refers to the ability of different healthcare systems, applications, and devices to exchange, interpret, and use clinical information seamlessly. It ensures that patient data flows efficiently across diverse systems and stakeholders (e.g., hospitals, clinics, pharmacies, laboratories) to improve care coordination, enhance decision-making, and support better patient outcomes.

However, achieving effective integration of clinical information across diverse systems comes with multiple challenges. In particular, clinical information comes from various disparate information systems, such as electronic medical record (EMR) systems, electronic health record (EHR) systems, laboratory systems, imaging systems, real-time patient monitoring devices, and others. These systems often use different data models and file formats, making it difficult to align data structures. In addition, the data models and file formats employed by different hospital systems also vary. For instance, shared clinical data from one hospital to another often has different structures, different healthcare identifiers, and different codes, which is problematic for any cross-content integration project. Despite standardization efforts like Health Level Seven (HL7®⁾ and Fast Healthcare Interoperability Resources (FHIR®), not all systems fully comply with or support these standards or provide a limited data set via these standards. For example, some EMR vendors design their systems to work primarily within their ecosystem, limiting interoperability.

### SUMMARY

The following presents a simplified summary of the specification in order to provide a basic understanding of some aspects of the specification. This summary is not an extensive overview of the specification. It is intended to neither identify key or critical elements of the specification, nor delineate any scope of the particular implementations of the specification or any scope of the claims. Its sole purpose is to present some concepts of the specification in a simplified form as a prelude to the more detailed description that is presented later.

According to an embodiment, a system includes at least one memory that stores computer-executable components, and at least one processor that executes the computer-executable components stored in the at least one memory. The computer-executable components can comprise a reception component that receives a bundle of healthcare resources configured in accordance with a Fast Healthcare Interoperability Resources (FHIR®) standard. The computer-executable components can further comprise a tracking database that tracks information regarding existing healthcare resources stored in a FHIR datastore, wherein the information comprises logical identifiers for the existing healthcare resources associated with the existing healthcare resources in the FHIR datastore and healthcare identifiers for the existing healthcare resources. The computer-executable components can further comprise a data refinement component that processes the bundle using a data refinement process and the tracking database and updates the tracking database to comprise new information extracted from the bundle.

In some embodiments, the computer-executable components further comprise a storage component that sends the bundle or a refined version of the bundle to the FHIR datastore following the data refinement process.

In various embodiments, the data refinement process comprises determining whether each resource of the healthcare resources corresponds to an existing healthcare resource stored in the FHIR datastore based on comparison of extracted healthcare identifiers for each resource to the healthcare identifiers for the existing healthcare resources in the tracking database. In some implementations, of these embodiments, based on a determination that a resource of the healthcare resources does not correspond to an existing healthcare resource, the data refinement process comprises maintaining the resource with the bundle and updating the tracking database to comprise a new entry for the resource.

In other implementations of these embodiments, based on a determination that a resource of the healthcare resources corresponds to an existing healthcare resource, the data refinement process comprises extracting an existing logical identifier for the resource from the tracking database, replacing a new logical identifier associated with the resource with the logical identifier, resulting in an updated version of the resource, and maintaining the updated version of the resource with the bundle, resulting in generation of a refined version of the bundle. In some implementations, the storage component can further send the refined version of the bundle to the FHIR datastore following the data refinement process. In addition, based on the determination that the resource of the corresponds to the existing healthcare resource, the data refinement process further comprises updating all (or in some implementations one or more) other references to the resource in the bundle with the existing logical identifier.

In some embodiments, the information in the tracking database further comprises sequence identifiers for the existing healthcare resources and source identifiers for the existing healthcare resources.

In accordance with these embodiments, based on a determination that a resource of the healthcare resources corresponds to an existing healthcare resource, the data refinement process comprises determining whether a source identifier of the source identifiers corresponding to the existing healthcare resource corresponds to a new source identifier associated with the resource. Based on a second determination that the source identifier does not correspond to the new source identifier, the data refinement process further comprises determining whether the new source identifier has a higher weight than the source identifier. The data refinement process further comprises based on a third determination that the new source identifier does not have a higher weight than the source identifier, removing the resource from the bundle, resulting in generation of a refined version of the bundle.

Additionally, or alternatively, the data refinement process further comprises, based on a third determination that the new source identifier has a higher weight than the source identifier, determining whether a new sequence identifier associated with the resource is higher than a sequence identifier of the sequence identifiers corresponding to the existing healthcare resource in the tracking database. In some implementations, based on a fourth determination that the new sequence identifier is not higher, removing the resource from the bundle, resulting in generation of a refined version of the bundle. In other implementations, based on a fourth determination that the new sequence identifier is higher, the data refinement process comprises maintaining the resource with the bundle.

In some embodiments, elements described in connection with the disclosed systems can be embodied in different forms such as a computer-implemented method, a computer program product, or another form.

For example, in another embodiment, a computer-implemented method, can comprise: receiving, by a system comprising a processor, a bundle of healthcare resources configured in accordance with a Fast Healthcare Interoperability Resources (FHIR®) standard; tracking, by the system, information regarding existing healthcare resources stored in a FHIR datastore in a tracking database, wherein the information comprises logical identifiers for the existing healthcare resources associated with the existing healthcare resources in the FHIR datastore and healthcare identifiers for the existing healthcare resources; and processing, by the system, the bundle using a data refinement process and the tracking database, wherein the data refinement process comprises updating the tracking database to comprise new information extracted from the bundle.

In another embodiment, a non-transitory machine-readable storage medium can comprise executable instructions that, when executed by a processor, facilitate performance of operations, comprising: receiving a bundle of healthcare resources configured in accordance with a Fast Healthcare Interoperability Resources (FHIR®) standard; tracking information regarding existing healthcare resources stored in a FHIR datastore in a tracking database, wherein the information comprises logical identifiers for the existing healthcare resources associated with the existing healthcare resources in the FHIR datastore and healthcare identifiers for the existing healthcare resources; and processing the bundle using a data refinement process and the tracking database, wherein the data refinement process comprises updating the tracking database to comprise new information extracted from the bundle.

### BRIEF DESCRIPTION OF THE DRAWINGS

Numerous aspects, implementations, objects and advantages of the present invention will be apparent upon consideration of the following detailed description, taken in conjunction with the accompanying drawings, in which like reference characters refer to like parts throughout, and in which:

FIG. 1 illustrates a high-level diagram of an example clinical data ingestion and normalization pipeline, in accordance with one or more embodiments described herein;

FIG. 2 illustrates a high-level block diagram of an example system that facilitates clinical data ingestion and normalization of proprietary formatted clinical data, in accordance with one or more embodiments described herein;

FIG. 3 illustrates a flow diagram of an example process for converting clinical data in a proprietary format into FHIR resources, in accordance with one or more embodiments described herein;

FIG. 4 illustrates an example mapping script for normalizing proprietary formatted clinical data, in accordance with one or more embodiments described herein;

FIG. 5 illustrates the relationship between an example mapping script and an example clinical data identifier script, in accordance with one or more embodiments described herein;

FIG. 6 illustrates example mapping data for normalizing proprietary formatted clinical data, in accordance with one or more embodiments described herein;

FIG. 7 illustrates an example computer-implemented method for normalizing proprietary formatted clinical data, in accordance with one or more embodiments described herein;

FIG. 8 illustrates a high-level block diagram of an example system that facilitates data deduplication management and clinical data identity resolution, in accordance with one or more embodiments described herein;

FIG. 9 illustrates a high-level flow diagram of an example process that facilitates data deduplication management and clinical data identity resolution, in accordance with one or more embodiments described herein;

FIG. 10 illustrates an example tracking database, in accordance with one or more embodiments described herein;

FIG. 11 illustrates a high-level flow diagram of an example process that facilitates racing condition handling, in accordance with one or more embodiments described herein;

FIG. 12 illustrates a high-level flow diagram of another example data refinement process, in accordance with one or more embodiments described herein;

FIG. 13 illustrates an example computer-implemented method that facilitates data deduplication management and clinical data identity resolution, in accordance with one or more embodiments described herein;

FIG. 14 illustrates an example computer-implemented method that facilitates racing condition handling, in accordance with one or more embodiments described herein;

FIG. 15 is a schematic block diagram illustrating a suitable operating environment; and

FIG. 16 is a schematic block diagram of a sample-computing environment.

### DETAILED DESCRIPTION

The following detailed description is merely illustrative and is not intended to limit embodiments and/or application or uses of embodiments. Furthermore, there is no intention to be bound by any expressed or implied information presented in the preceding Background section, Summary section or in the Detailed Description section.

The disclosure describes a cloud-based clinical data processing pipeline solving many challenges associated clinical data integration, including how to consume and harmonize massive amounts of heterogonous clinical data received over time in parallel.

The disclosure describes techniques to harmonize and ingest all received heterogonous data in one single format, the FHIR format.

Fast Healthcare Interoperability Resources (FHIR®) is a standard developed by HL7 (Health Level Seven International) to facilitate the exchange of healthcare information electronically. FHIR is designed to enable interoperability between different healthcare systems and is widely used for building modern healthcare applications. It combines existing healthcare data standards with web technologies to make data sharing more accessible and efficient.

The proposed pipeline provides several solutions. In particular, the proposed pipeline provides for normalizing heterogonous structured clinical data into one unique model (FHIR based model). The proposed pipeline further enriches the clinical data with codes and concepts normalization and indexation. The proposed pipeline further resolves duplications of data coming from different sources. Finally, the proposed pipeline provides a high-performance method to resolve data racing conditions.

The data ingestion and normalization pipeline provides numerous technical advantages. In particular, the pipeline enables data manipulation and exploitation by third party products, like command centers, clinical applications, and others. By normalizing the data to one unique output structure, the pipeline enables efficient software development of new technologies consuming FHIR resources. Data enrichment with standardized codes further enables downstream applications and entities (e.g., hospitals, clinicians, researchers, etc.) to search and access data the same way within different deployment scenarios or domains. The proposed pipeline further enables data capacities to improve clinical information processing algorithms through artificial intelligence (AI) and accelerates patient access to healthcare while improving intervention outcomes for patient care.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

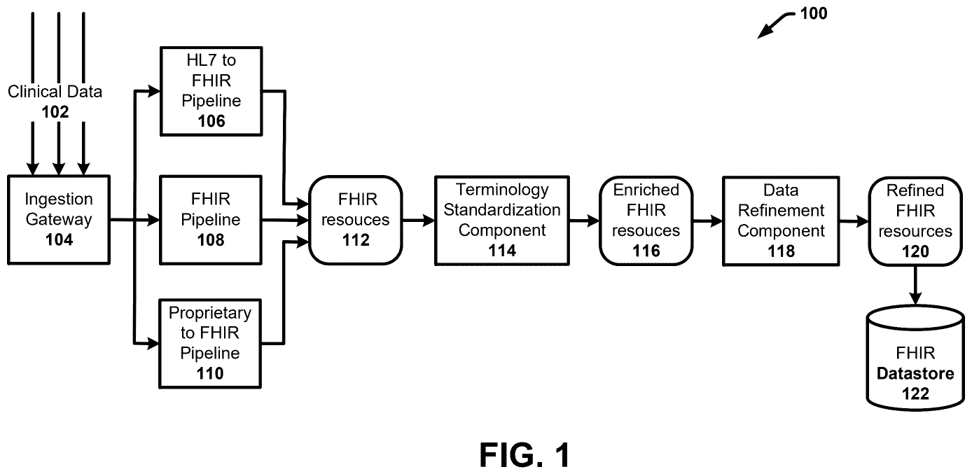

Turning now to the drawings, FIG. 1 illustrates an example clinical data ingestion and normalization pipeline 100, in accordance with one or more embodiments described herein. Pipeline 100 can include or correspond to one or more computing devices, machines, virtual machines, computer-executable components, datastores, and the like that may communicatively be coupled to one another either directly or via one or more wired or wireless communication frameworks. For example, pipeline 100 can include ingestion gateway 104, HL7 to FHIR pipeline 106, FHIR pipeline 108, proprietary to FHIR pipeline 110, terminology standardization component 114, data refinement component 118 and FHIR datastore 122. These elements can respectively include or correspond to one or more computing devices, machines, virtual machines, computer-executable components, datastores, and the like that may communicatively be coupled to one another either directly or via one or more wired or wireless communication frameworks.

Pipeline 100 facilitates ingesting clinical data 102 in various formats from various clinical information systems, normalizing the clinical data 102 into the FHIR format, and storing the clinical data in the FHIR format in a FHIR datastore 122. Pipeline 100 involves five main processes, including data ingestion, data structure normalization, data enrichment, data refinement, and storage. In this regard, the first process of pipeline 100 involves the ingestion of multiple kinds of clinical data 102 via ingestion gateway 104 as received from various clinical information systems and in various formats. Clinical data 102 thus includes or corresponds to heterogenous clinical data. For example, the clinical information systems can include various disparate types of electronic healthcare information systems, such as EHR systems, imaging systems, laboratory systems, medical devices, patient monitoring devices, bed management systems, administrative systems (e.g., billing systems, scheduling systems, etc.), and various others. The healthcare information systems can be associated with the same healthcare enterprise (e.g., the same hospital or the hospital enterprise) and/or disparate healthcare enterprises. Clinical data 102 can include data pushed to the ingestion gateway 104 from these disparate healthcare information sources regularly or continuously over time as it is generated (e.g., in real-time) or in response to another defined event or condition. In this regard, clinical data 102 can include or correspond to a stream of clinical data that is regularly or continuously received by the ingestion gateway 104 from various clinical information sources over time.

In accordance with various embodiments, the ingestion gateway 104 includes or corresponds to an application program interface (API) that categorizes the clinical data 102 into one of three types of clinical data based on the format in which it is received. These three types include HL7 clinical data in the HL7 format, FHIR clinical data in the FHIR format, and clinical data in any other proprietary format other than HL7 or FHIR. In various embodiments, the proprietary format can include a JSON format. Additionally, or alternatively, the proprietary format can include a CSV format. These three different types of clinical data are respectively normalized through separate normalization pipelines. In this context, normalization refers to converting the data into the FHIR format. For example, the ingestion gateway 104 sends HL7 clinical data to the HL7 to FHIR pipeline which converts the HL7 clinical data into to the FHIR format. The ingestion gateway 104 further sends clinical data received in the FHIR format through the FHIR pipeline 108 which ensures the data is structured correctly in accordance with the FHIR format. The ingestion gateway 104 further sends clinical data received in a proprietary format, such as CSV files and JSON files, through the proprietary to FHIR pipeline 110, which converts the clinical data from the proprietary format into the FHIR format. Thus, the output of these three respective pipelines is normalized representations of the clinical data 102 in the FHIR format. In other words, following normalization via the respective pipelines 106, 108 and 110, respective data messages, files, data objects, and the like, included in the clinical data 102 are converted into FHIR resources 112 in accordance with the FHIR format.

In this regard, the FHIR format organizes healthcare data into modular units called resources. FHIR resources are the building blocks of the FHIR standard and represent key entities and concepts in healthcare. Each FHIR resource is a structured, reusable, and standardized representation of a specific piece of healthcare data. These resources can be combined and extended to cover various healthcare use cases, including patient records, clinical workflows, and public health reporting. Each resource contains a well-defined set of data that represents a specific concept, such as a patient, an observation, a medication, and various other concepts. FHIR resources are categorized based on their purpose. Some of the key categories include clinical, administrative, financial, infrastructure and others. Clinical FHIR resources represent clinical data and workflows. Some examples of clinical FHIR resources include: a patient resource, which may include demographic data about a patient; a condition resource that includes data regarding a clinical condition or diagnosis; an observation resource that may include physiological measurements or test results (e.g., blood pressure); and an allergy intolerance resource that includes information about patient allergies. Administrative resources pertain to non-clinical workflows, like scheduling and billing. Some examples of administrative resources include: a practitioner resource, which includes information about a healthcare professional; and organization resource that includes data about healthcare organizations; an appointment resource, that includes scheduling and booking information; and an encounter resource that includes details of a patient visit or interaction. Financial resources support billing, payments, and claims processing. Some examples of financial resources include: a claim resource, which includes information pertaining to a request for payment for services, and a coverage resource, which includes details about insurance or payment agreements. Infrastructure resources support system-level operations and data exchange.

FHIR currently has defined more than 140 resources. Each FHIR resource has a standard structure, including mandatory and optional fields, making it flexible for diverse use cases. For example, every resource includes key fields, including a unique identifier (ID) for the resource, metadata about the resource (e.g., version, source, last updated, etc.), and data elements or core fields specific to the resource. Resources can also be extended to meet specific local or organizational needs using extensions, allowing additional fields while maintaining compatibility with the base standard. FHIR resources can be represented in JSON, XML, or Turtle (RDF) formats for interoperability. Resources are designed to work seamlessly with RESTful APIs, enabling CRUD (Create, Read, Update, Delete) operations using standard hypertext transfer protocol (HTTP) methods like GET, POST, PUT, and DELETE. FHIR resources provide the foundation for a flexible and interoperable healthcare ecosystem. Their modular design, extensibility, and alignment with web standards make them a powerful tool for modern healthcare information technology solutions.

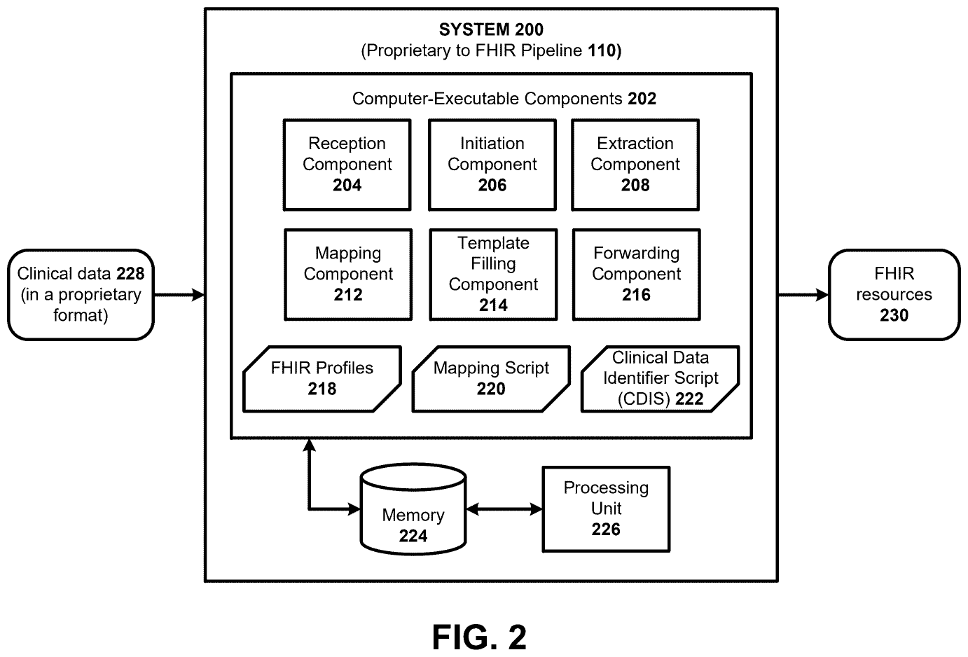

In various embodiments, the HL7 to FHIR mapping can be performed by the HL7 to FHIR pipeline 106 through different commercial or proprietary tools, like Microsoft converter®, Google converter®, IBM converter®, or another proprietary converter. The FHIR pipeline 108, upon reception of FHIR data, performs only sanitization and validation of the data. The proprietary to FHIR pipeline 110 converts clinical data in a proprietary format such as CSV and/or JSON format, into FHIR resources 112 through a proprietary mapping process between these proprietary file formats and the FHIR resources. This proprietary mapping process is described in greater detail infra with reference to FIGS. 2-7.

Upon generation of the FHIR resources 112, regardless of the pipeline through which they are generated (e.g., the HL7 to FHIR pipeline 106, the FHIR pipeline 108, or the proprietary to FHIR pipeline 110), the terminology standardization component 114 performs a terminology standardization process to convert the FHIR resources 112 into enriched FHIR resources 116. The terminology standardization process involves mapping terms included in the FHIR resources 112 to corresponding standard terms in accordance with a standardized ontology or nomenclature. For example, some hospitals may use their own coding system with unique terms and codes to reference particular data elements. The terminology standardization process matches these unique terms and codes to their corresponding standardized versions of these terms and replaces the unique terms and codes with their corresponding standardized versions. This process essentially corresponds to translating a term in a first language to the same term in a second, standardized language. The terminology standardization component 114 can perform this process using one-to-one mapping and/or preconfigured AI translation algorithms for code/term translation. Data enrichment with normalized codes allows consuming applications to search and access data the same way within different deployment environments or domains.

The data refinement component 118 further refines the enriched FHIR resources to convert the enriched FHIR resources 116 into refined FHIR resources 120. The refined FHIR resources 120 are then stored in the FHIR datastore 122. The data refinement performed by the data refinement component 118 involves processing the enriched FHIR resources to ensure the same resource is not duplicated more than once in the FHIR datastore 122, a process referred to herein as data de-duplication. As used herein, data de-duplication refers to the process of identifying and resolving duplicate instances of FHIR resources within the refined FHIR resources and the FHIR datastore 122. In healthcare, duplicate data often occurs due to fragmented systems, repeated data entry, or the integration of data from multiple sources. De-duplication ensures that information is accurate, consistent, and non-redundant, improving data quality and system efficiency. Data de-duplication is an essential piece of pipeline 100. The data de-duplication process performed by the data refinement component 118 eliminates inconsistencies caused by duplicate entries, ensuring that healthcare providers and systems work with reliable data. This reduces confusion when exchanging data between systems by ensuring there is only one authoritative version of each resource. In addition, the data de-duplication process reduces storage requirements of the FHIR datastore by avoiding storage of redundant data. Furthermore, by ensuring each resource in the FHIR datastore is not duplicated, downstream errors caused by conflicting or redundant records (e.g., duplicate medication orders) are eliminated.

In some embodiments, in association with performing data de-duplication, the data refinement component 118 also performs racing condition management. Race conditions in FHIR refer to situations where the outcome of operations on FHIR resources depends on the sequence or timing of concurrent events. This typically occurs when multiple systems, users, or processes try to access or modify the same resource at the same time, leading to potential conflicts, data inconsistencies, or unexpected behavior. As applied to pipeline 100, racing conditions can occur when updates to the same FHIR resource are received through the pipeline 100 at or near the same time yet from disparate sources. For example, one healthcare source corresponding to a practitioner's information technology system may provide an update to a patient's demographic details, while another information system concurrently sends an update to the patient's address through the pipeline, potentially overwriting data. Racing conditions can also occur when two operations depend on the state of a resource, and their outcomes conflict due to timing. For example, a prescription resource may be received for a patient at the same time a status update for the patient is received marking the patient as inactive or deceased. Racing conditions can also occur when one process creates a resource while another process simultaneously attempts to reference it, leading to failures if the resource isn't yet available. The consequences of racing conditions observed through pipeline 100 can include data overwriting in the FHIR datastore 122, wherein critical updates may be unintentionally overwritten by conflicting changes, and data loss, wherein valuable information might be erased or replaced in the FHIR datastore 122 due to uncoordinated actions. In addition, resources may end up in invalid or incomplete states, leading to potential errors downstream.

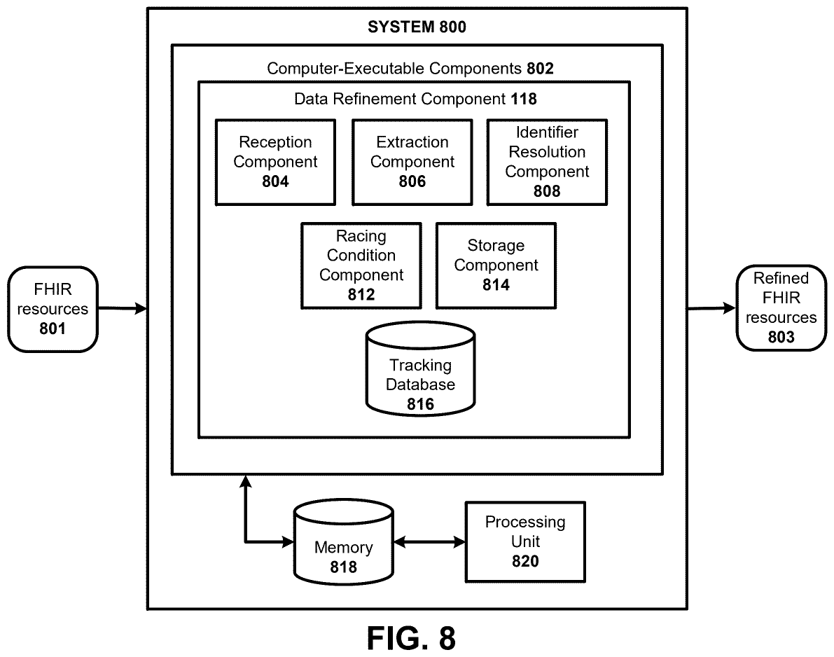

The disclosed subject matter is particularly directed to the proprietary to FHIR pipeline 110 and the data refinement component 118. The features and functionalities of the proprietary to FHIR pipeline 110 are described in greater detail with reference to FIGS. 2-7, and the features and functionalities of the data refinement component 118 are described in greater detail with reference to FIGS. 8-13.

FIG. 2 illustrates a high-level block diagram of an example system 200 that facilitates clinical data ingestion and normalization of proprietary formatted clinical data, in accordance with one or more embodiments described herein. With reference to FIGS. 1 and 2, in various embodiments, system 200 corresponds to proprietary FHIR pipeline 110. Aspects of the systems, apparatuses or processes explained in this disclosure can constitute computer-executable or machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described.

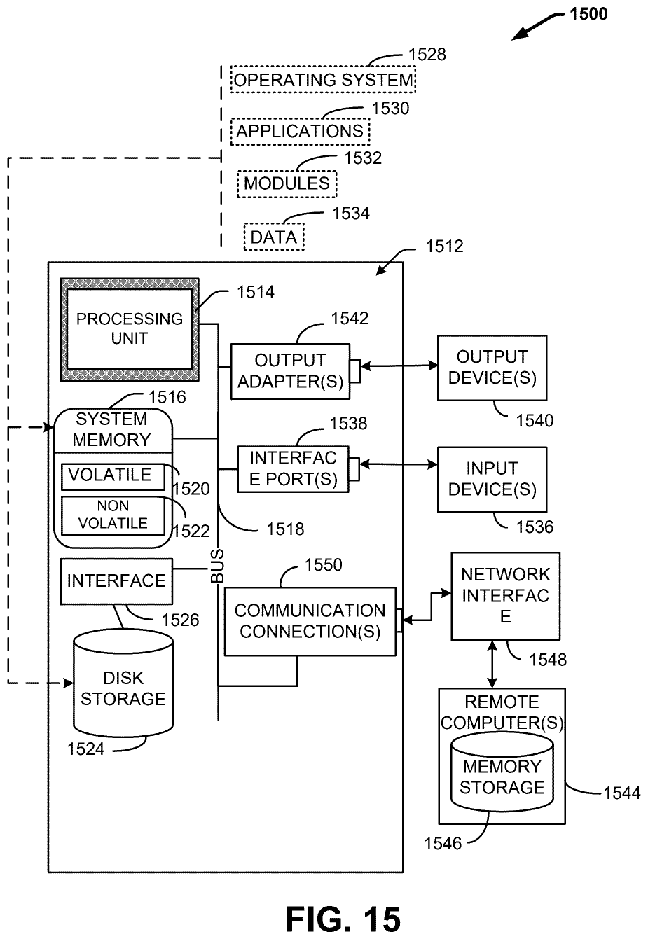

For example, system 200 can comprise at least one memory 224 that stores computer-executable components 202, and at least one processor or processing unit 226 that executes the computer-executable components 202 stored in the at least one memory 224. The computer-executable components 202 can include, but are not limited to, reception component 204, initiation component 206, extraction component 208, mapping component 212, template filling component 214, forwarding component 216, FHIR profiles 218, mapping script 220 and clinical data identifier script (CDIS) 222. In various embodiments, computer-executable components 202 correspond to proprietary to FHIR pipeline 110. In other words, proprietary FHIR pipeline 110 can include or correspond to a computer-executable component comprising sub-components including reception component 204, initiation component 206, extraction component 208, mapping component 212, template filling component 214, forwarding component 216, FHIR profiles 218, mapping script 220 and CDIS 222. Examples of said memory 224 and processing unit 226 as well as other suitable computer or computing-based elements, can be found with reference to FIG. 15 (e.g., system memory 1516 and processing unit 1514 respectively), and can be used in connection with implementing one or more the components shown and described in connection with FIG. 2, or other figures disclosed herein.

The reception component 204 receives clinical data 228 (e.g., from the ingestion gateway 104) wherein the clinical data 228 is in a proprietary format. In some embodiments, the proprietary format can include a JSON format. In other embodiments, the proprietary format can include a non-JSON format, such as a CSV format. In embodiments in which the proprietary format does not include a JSON format, the initiation component 206 translates the clinical data from the non-JSON format into the JSON format prior to transforming the clinical data 228 into FHIR resources 230. The clinical data 228 may be structured or unstructured in accordance with the proprietary format. The clinical data 228 can correspond to a data message, a JSON file, a CSV file or the like. In this regard, although various features and functionalities of the computer-executable component 202 are described in association with converting a single file or message into one or more FHIR resources 230, it should be appreciated that system 200 is configured to receive and process a continuous stream of clinical data 228 corresponding to messages or files regularly or continuously received over time. In this context, the clinical data 228 may be received from various disparate healthcare information systems over time.

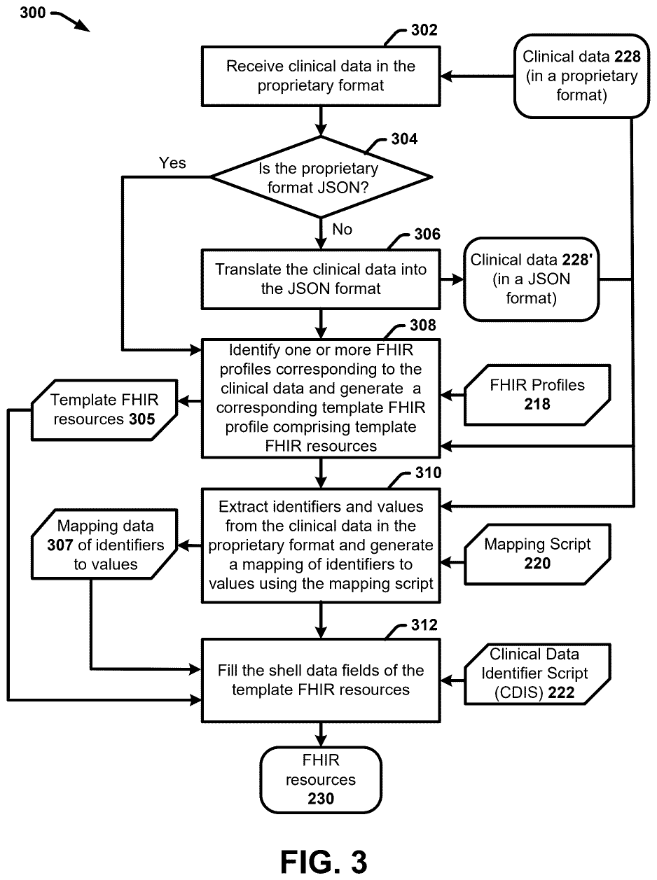

As noted above, the computer-executable components 202 perform a conversion process to convert the clinical data 228 into one or more FHIR resources 230 in accordance with the FHIR format. The one or more FHIR resources 230 can include or correspond to one or more of the FHIR resources 112 coming out of the proprietary to FHIR pipeline 110. At a high-level, this conversion process preformed by the proprietary to FHIR pipeline 110 involves converting the clinical data in the proprietary format into the FHIR resources 230 based on mapping data elements extracted from the clinical data to one or more FHIR profiles defined for the clinical data. This conversion process and the corresponding features and functionalities of the computer-executable components 202 are described with reference to FIG. 3.

FIG. 3 illustrates a flow diagram of an example process 300 for converting clinical data 228 in a proprietary format into FHIR resources 230, in accordance with one or more embodiments described herein. With reference to FIGS. 2 and 3, process 300 begins with the reception of clinical data 228 in the proprietary format at 302 (e.g., via reception component 204). Following reception of the clinical data 228, at 304, if the proprietary format is a JSON format, then process 300 proceeds to 308. However, if the proprietary format is not a JSON format, such as a CSV format or another type of non-JSON format, then at 306, the initiation component translates the clinical data into the JSON format, resulting in clinical data 228′ in the JSON format.

At 308, the initiation component 206 first identifies one or more FHIR profiles included in FHIR profiles 218 corresponding to the clinical data 228 (or clinical data 228′) and generates a corresponding template FHIR profile comprising template FHIR resources 305.

FHIR profiles are customized definitions of FHIR resources that specify how those resources should be used in specific contexts or for specific use cases. They extend or constrain the base FHIR standard to meet the requirements of a particular organization, project, or region, ensuring consistent data exchange and interpretation. For example, as noted above, FHIR currently defines more than 140 different resources respectively corresponding to different categories of healthcare information, including clinical and non-clinical information (e.g., administrative, financial, etc.). These base definitions resources define the format in which the FHIR resource is represented and define the required data elements for each FHIR resource and optional data elements for each resource. Profiles extend the FHIR standard by adding custom elements using extensions. They also constrain the base FHIR resources by restricting allowed values, mandating specific fields (e.g., making birthDate a required field for a Patient resource) and addition additional rules or business logic.

For example, an Observation is one type of FHIR resource included amongst the different types of FHIR resources. There are many types of clinical data that can be considered as an observation. For example, vital signs such as pulse rate, blood pressure, and temperature can be different types of observations. In another example, laboratory data like blood glucose, imaging results, clinical findings, device measurements, personal characteristics such as eye-color, and various other can also be types of observations. The FHIR resource type Observation defines a JSON template to be used for every observation and includes required data fields and constraints on values or text that can be included in each data field. Profiles are used for example to further refine FHIR resources classified as Observations into various sub-types tailored to particular use cases. In this regard, a FHIR profile is a restriction of the generic FHIR resource type, by adding some constraints to align the resource to the use case. For example, a vital sign is a kind of Observation, but needs to have extra restrictions, like the category of the observation shall have a fixed value: “vital-signs”. In another example, a Location is another type of FHIR resource included amongst the different defined FHIR resource types. A hospital might define custom profiles for the Location resource to define different types of Location resources, such as a bed location, a room location, and a facility location.

In this regard, FHIR profiles 218 include or correspond to a list of predefined FHIR profiles, which may be tailored to a particular organization, project, or region. In other words, the FHIR profiles 218 can include tailored sub-definitions of various FHIR resources, wherein each profile defines the required and fixed elements and optional elements and constraints on the data (i.e., the values, the text, etc.) that can be included in the FHIR resource.

Generally, the respective FHIR profiles 218 represent a single FHIR resource. In other words, a single FHIR profile typically defines constraints for a single resource. For example, a custom Patient profile might specify: mandatory fields like identifier and birthDate, restrictions on the values for gender, and extensions for additional fields like “preferred language”. This custom profile applies only to the Patient resource type. However, FHIR profiles 218 can also reference other profiles to ensure consistent relationships between resources. For example, a custom profile for an Observation might require references to specific profiles for related resources, such as: a Patient profile for the Observation.subject, or a Practitioner profile for the Observation.performer. This creates a network of profiles covering multiple resources. For example, suppose the FHIR profiles 218 includes multiple profiles related to the use case of clinical data 228 that reports laboratory results. The multiple profiles may include a first profile for the Observation resource that requires the Observation.code to use a specific laboratory code. The same profile may also mandate a reference to a patient profile. Accordingly, the FHIR profiles would also contain a separate Patient profile with specific constraints. Together, these profiles ensure that lab results conform to the Observation profile and that the patient referenced in the results conforms to the Patient profile.

In accordance with process 300, at 308, the initiation component 206 identifies one or more FHIR profiles included amongst the FHIR profiles 218 corresponding to the clinical data 228 (or clinical data 228′). This involves identifying terms or elements included in the clinical data corresponding to one or more defined profiles of amongst the FHIR profiles. For example, let's assume the clinical data 228 corresponds to a bed request message in a JSON format. In FHIR, a bed request typically corresponds to a situation where a patient requires assignment to a hospital bed, often as part of admission or care management processes. While FHIR does not have a dedicated resource specifically named “BedRequest,” this concept can be modeled using a combination of existing FHIR resources as tailored using one or more custom FHIR profiles depending on the context and requirements of the workflow. The bed request message for example may include a message identifier indicating the type of message is a bed request and the FHIR profiles may include a specific profile for bed requests, that also accounts for a combination of different FHIR resources, such a ServiceRequest resource, a Patient resource, a location resource, and the like.

In most embodiments, the clinical data 228 will reference a collection of multiple FHIR resources. A collection of FHIR resources is referred to as a FHIR bundle. In some implementations, a single FHIR profile can be defined for a FHIR bundle. With these implementations, the template FHIR resources 305 created at 308 can represent multiple FHIR resources. In other implementations, at 308, the initiation component 206 can identify multiple FHIR profiles referenced in the clinical data and create templates FHIR resources for each of the multiple FHIR profiles. In either of these cases, the one or more template FHIR resources 305 correspond to structured JSON files with data fields including one or more fixed values (as defined via the FHIR profile, such as the fixed value “vital signs” for an Observation category field) and one or more shell data fields that need to be filled with text and/or values extracted from the clinical data 228 (or clinical data 228′). In other words, the FHIR profiles 218 are used to initiate the generated FHIR resources 305 with required and fixed elements. For example, if the FHIR profile selected at 308 is the VitalSign profile, the template FHIR resource may include many elements, including a fixed element for the category element that will contain the value “vital-sign,” and other shell data fields that need to be filled, such as a required field referencing the patient involved and the like.

At 310, the extraction component 208 extracts identifiers of clinical data elements and corresponding values from the clinical data 228 (or clinical data 228′) and the mapping component 212 generates mapping data 307 of the identifiers to the values. The identifiers correspond to identifiers of data elements corresponding to shell data fields that need to be filled, and the values correspond to the actual values to be included in the corresponding shell data fields. To facilitate this end, the extraction component 208 and the mapping component 212 employs mapping script 220. At 312, based on the CDIS 222, the mapping data 307 and the template FHIR resource 305, the template filling component 214 then fills the shell data fields of the template FHIR resource 305 with the extracted clinical data to generate the FHIR resources 230.

As noted above, FHIR resources 230 correspond to the portion of the FHIR resources 112 flowing out of the proprietary to FHIR pipeline 110. In various embodiments, the forwarding component 216 sends these FHIR resources 230 (and/or FHIR resource 112) to terminology standardization component 114 which then transforms them into the enriched FHIR resources 116.

In this regard, the CDIS 222 allows the template filling component 214 to identify non-fixed values in the FHIR profiles with specific identifiers to be used. For example, as applied to a resource for a custom Observation profile corresponding to a vital sign, the “effectiveDateTime” may be defined in the profile as an element that needs to be collected from the proprietary clinical data structure. The CDIS 222 is a JSON file containing Key-Values elements, including identifiers, and the JSON path to the FHIR resources. For example, for the vital sign date, the key-value might be: (DATETIME, Observation.effectiveDateTime).

The mapping script 220 enables the mapping component 212 to map clinical data between the proprietary format to the identifiers of clinical data described in the CDIS 222, another JSON document. The mapping script is also a JSON file with Key-Value elements, where the Keys are the identifiers from the CDIS document, and the value is a JSON path describing the link to the proprietary data.

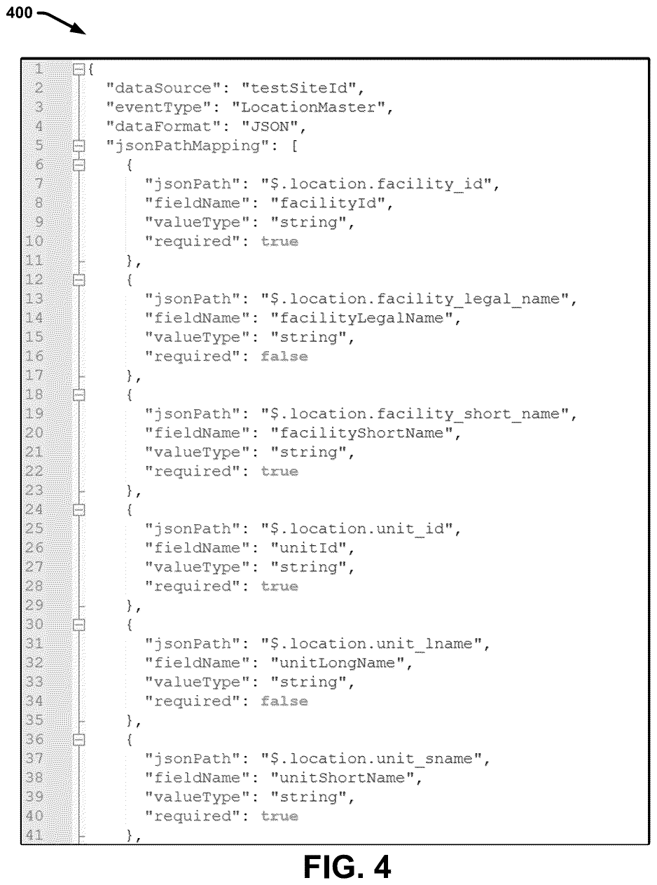

For example, FIG. 4 illustrates an example of a portion of a mapping script 400 in accordance with one or more embodiments described herein. In this example, the mapping script 400 is a JSON file that identifies clinical data elements via identifiers. The identifiers of the keys correspond to the field name. For instance, in this example, the key data elements are applicable to a bedRequest FHIR profile, and include information such as the facility ID, the facility legal name, the facility short name, the unit ID, the unit long name, and others. In addition to the field names, the mapping script also include other key elements associated with the clinical data elements, including the value type and whether the data element is required or not for the corresponding FHIR resource. Furthermore, the mapping script provides the JSON Path for each data element corresponding to a fieldname in the proprietary format. In this regard, in order to identify the corresponding values for the respective identifiers (i.e., the field names), the extraction component 208 executes the JSON path within the property clinical data structure. In other words, at 310 of process 300, for each field name included in the mapping script, the extraction component 208 executes corresponding JSON path in order to identify and extract the corresponding values from the proprietary clinical data 228, such as the actual facility ID, the actual facility short name, and so on. The output at 310 thus includes a mapping of the identifiers in the mapping script (e.g., the field names, to the actual extracted values). The JSON paths for each data element depend on the proprietary clinical data structure. In this regard, the mapping script 400 (and mapping script 220) includes the JSON paths for all data elements received in the clinical data 228 in the proprietary format as identified by corresponding identifiers (i.e., the field names).

After the mapping data 307 has been generated, at 310, the values need to be mapped to the corresponding FHIR resource fields included in the template FHIR resources 305 in order to fill the shell data files with the values. To facilitate this end, the clinical data identifier script 222 provides a mapping between the identifiers and the corresponding FHIR resource fields included in the template FHIR resources.

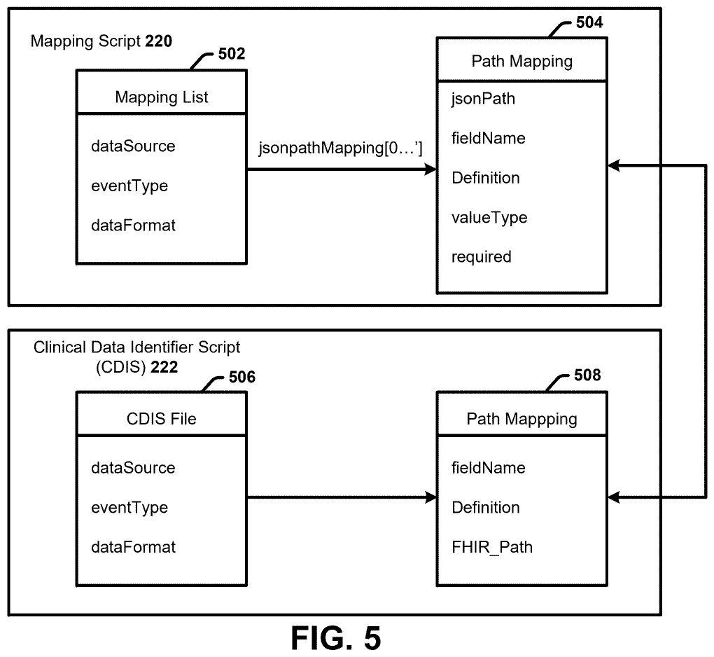

In this regard, FIG. 5 illustrates the relationship between the mapping script 220 and the CDIS 222, in accordance with one or more embodiments described herein. The mapping script 220 includes a mapping list 502 and path mapping information 504. The mapping list identifies the type of data elements that need to be extracted from the proprietary clinical data 228. The path mapping information 504 provides the JSON path for the respective elements included in the mapping list 502, the field name (which corresponds to the identifier for the data element), the definition of the data element, the value type and whether it is required.

CDIS 222 includes a CDIS file 506 that includes corresponding information for the path mapping 508. The path mapping 508 provides the FHIR path to the corresponding FHIR identifiers for respective field names included in the path mapping information 504 as well as the definition. The field name in the path mapping 508 from CDIS 222 refers to the field name element in path mapping 504 from the mapping script 220.

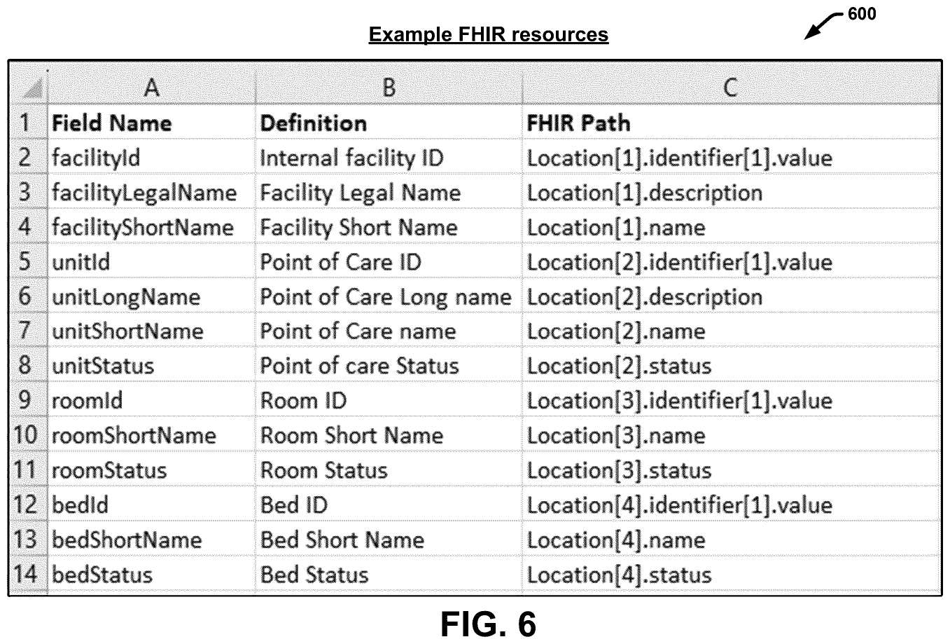

FIG. 6 presents a table 600 illustrating an example CDIS file in a simplified presentation, describing the elements of path mapping 508 related to CDIS file 506. In accordance with examples in table 600, column A includes the identifiers of the data elements that were extracted from the proprietary clinical data. Column B includes their definitions, and column C includes their corresponding FHIR paths, in relationship with the template FHIR resources 305.

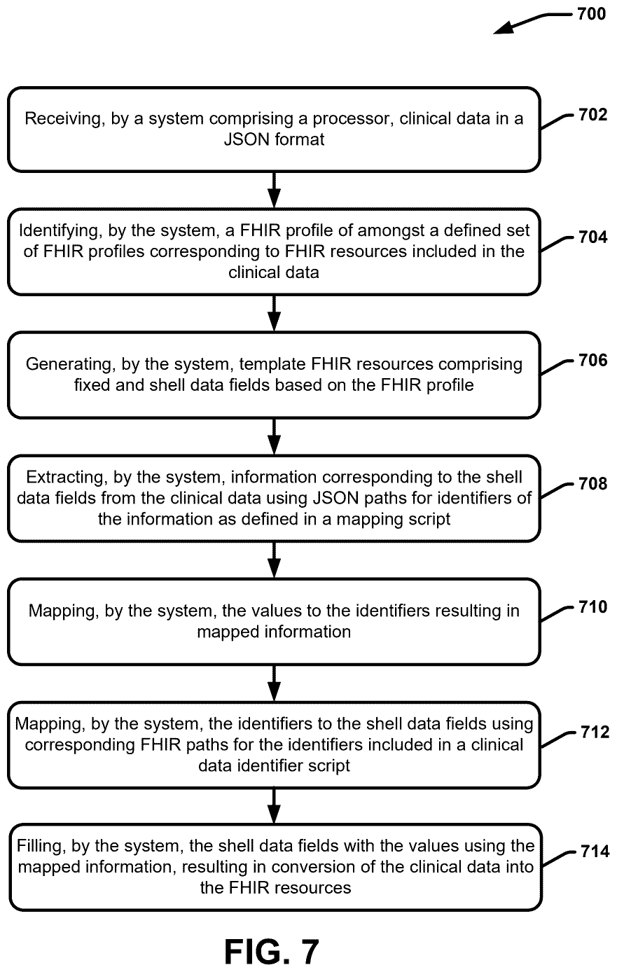

FIG. 7 illustrates an example computer-implemented method 700 for normalizing proprietary formatted clinical data, in accordance with one or more embodiments described herein. Method 700 corresponds to an example method that can be performed by system 200 in accordance with one or more embodiments. Method 700 comprises, at 702, receiving, by a system comprising a processor, clinical data in a JSON format (e.g., via reception component 204). For example, the proprietary format may be a JSON format, a CSV format, or another proprietary format other than the HL7 or the FHIR format. In implementations, in which the proprietary format is not JSON, the initiation component 206 converts the clinical data into the JSON format prior to converting the clinical data into FHIR resources, as noted in process 300. At 704, method 700 comprises identifying, by the system, a FHIR profile of amongst a defined set of FHIR profiles (e.g., FHIR profiles 218) corresponding to FHIR resources included in the clinical data (e.g., via initiation component 206). At 706, method 700 comprises generating, by the system, template FHIR resources comprising fixed and shell data fields based on the FHIR profile (e.g., via initiation component 206). At 708 method 700 comprises extracting, by the system, information corresponding to the shell data fields from the clinical data using JSON paths for identifiers of the information as defined in a mapping script (e.g., via extraction component 208). At 710, method 700 comprises mapping, by the system, the values to the identifiers, resulting in mapped information (e.g., mapping data 307), (e.g., via mapping component 212). At 712, method 700 comprises mapping, by the system, the identifiers to the shell data fields using corresponding FHIR paths for the identifiers included in a clinical data identifier script. At 714, method 700 comprises filling, by the system, the shell data fields with the values using the mapped information, resulting in conversion of the clinical data into the FHIR resources (e.g., via template filling component 214).

FIG. 8 illustrates a high-level block diagram of an example system 800 that facilitates data deduplication management and clinical data identity resolution, in accordance with one or more embodiments described herein. In some embodiments, system 800 can include system 200, or vice versa. For example, in some embodiments, system 800 can include one or more of computer-executable components 202. With reference to FIGS. 1 and 8, aspects of the systems, apparatuses or processes explained in this disclosure can constitute computer-executable or machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described.

For example, system 800 can comprise at least one memory 818 that stores computer-executable components 802, and at least one processor or processing unit 820 that executes the computer-executable components 802 stored in the at least one memory 818. The computer-executable components 802 include data refinement component 118, which can include reception component 804, extraction component 806, identifier resolution component 808, racing condition component 812, storage component 814 and tracking database 816. Examples of said memory 818 and processing unit 820, as well as other suitable computer or computing-based elements, can be found with reference to FIG. 15 (e.g., system memory 1516 and processing unit 1514 respectively), and can be used in connection with implementing one or more the components shown and described in connection with FIG. 8, or other figures disclosed herein.

With reference to FIG. 8 in view of FIG. 1, in various embodiments, the reception component 804 receives FHIR resources 801 and the data refinement component 118 refines the FHIR resources 801 into refined FHIR resources 803, which are then sent to and stored in the FHIR datastore 122 (e.g., via storage component 814). In some embodiments, FHIR resources 801 can correspond to enriched FHIR resources 116 as generated by the terminology standardization component 114 in accordance with pipeline 100, and refined FHIR resources 803 can include or correspond to refined FHIR resources 120. Additionally, or alternatively, the FHIR resources 801 can include or correspond to FHIR resources 112 and the terminology standardization component 114 may be removed from pipeline 100.

As noted with reference to FIG. 1, a major issue that can occur when clinical data 102 is received through ingestion gateway 104 from different sources (and even the same source) in parallel over time involves reception of clinical data referring to an existing resource in the FHIR datastore 122. Often times, this can result in the FHIR resource being duplicated in the FHIR datastore 122 or reference to different instances of the same resource using different identifiers, resulting in confusion as to the correct representation of the data. In addition, as clinical data 102 is received in parallel through different pipelines (e.g., the HL7 to FHIR pipeline 106, the FHIR pipeline 108, and the proprietary to FHIR pipeline 110) pertaining to the same FHIR resource or an existing resource in the FHIR datastore 122, racing conditions often occur, resulting in errors such as inconsistency amongst the data, overriding of data, and others.

In various embodiments, the data refinement component 118 reviews and refines the FHIR resources 801 prior to sending them to the FHIR data store 122 (e.g., via storage component 814, as refined FHIR resources 120) to ensure the FHIR resources are not duplicated and to mitigate issues pertaining to racing conditions. In this regard, refinement of the FHIR resources 801 is a complicated process. The goal is to be able to handle clinical data coming from multiple heterogonous sources and avoid duplication of created resources in the FHIR datastore 122. Without resolving duplicated resources before pushing them to the FHIR datastore 122, data can get mixed up, duplicated, and related FHIR resources can become improperly linked. Thus, the data refinement component 118 is the cornerstone for any successful cross-sources integration project.

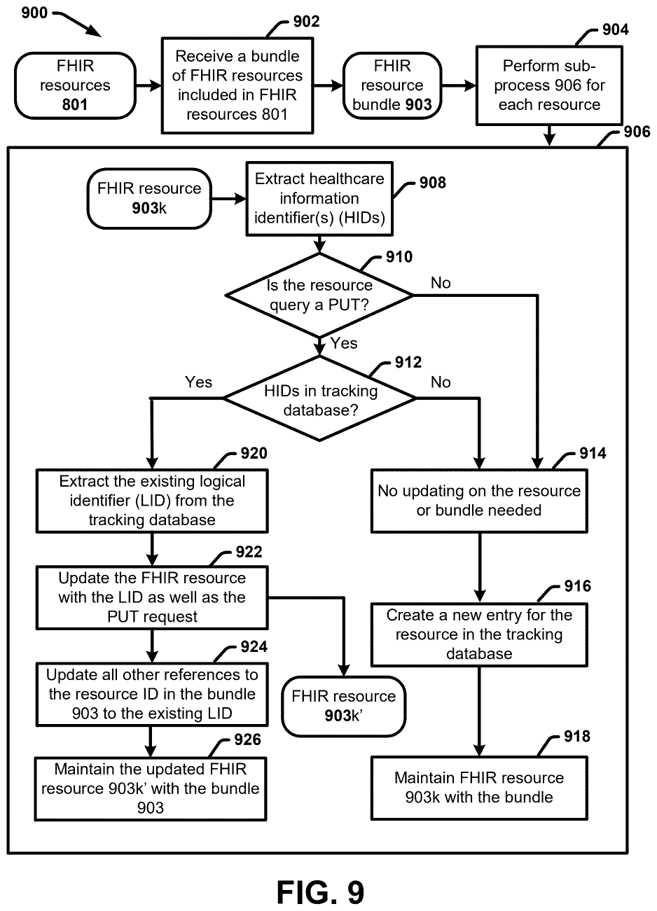

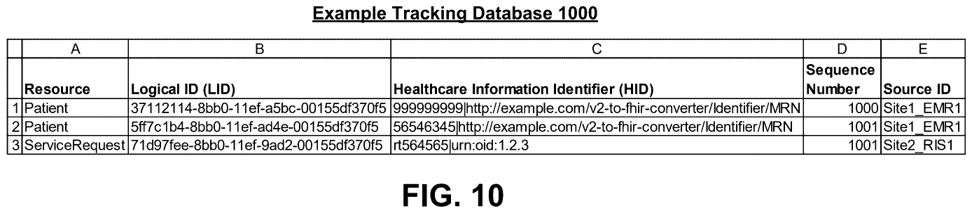

In various embodiments, the data refinement component 118 can perform data refinement process that facilitates data de-duplication and a clinical data identity resolution using extraction component 806, identifier resolution component 808, and tracking database 816. This data refinement process and the corresponding features and functionalities of these components as applied to this data refinement process are discussed with reference to FIGS. 9 and 10. Additionally, or alternatively, the data refinement component 118 can perform another data refinement process using extraction component 806, identifier resolution component 808, racing condition component 812 and tracking database 816. This data refinement process and the corresponding features and functionalities of these components as applied to this data refinement process are discussed with reference to FIG. 10.

Turning now to FIG. 9 in view of FIG. 8., FIG. 9 illustrates a high-level flow diagram of an example data refinement process (i.e., process 900) that facilitates data deduplication management and clinical data identity resolution, in accordance with one or more embodiments described herein. Process 900 begins at 902, wherein the reception component 804 receives a bundle of FHIR resources (FHIR resource bundle 903) included in the FHIR resources 801. In this regard, in some embodiments, the FHIR resources 801 can include individual FHIR resources. In other embodiments, the FHIR resources 801 can include groups of FHIR resources generated from the same clinical data message or file or group of clinical data messages/files and aggregated together as a FHIR bundle. For example, as described with reference to FIG. 3, often times, clinical data 228 may reference a plurality of FHIR resources, such as an observation, a patient, a location, etc. This applies to clinical data processed through the HL7 to FHIR pipeline 106 as well as the FHIR pipeline 108.

Process 900 is described in association with processing a bundle of FHIR resources, also referred to as a FHIR resource bundle 903. With these embodiments, process 900 assumes FHIR resources 801 includes a bundle of FHIR resources and at 904, sub-process 906 is performed for each resource in the bundle independently. In some embodiments, after every resource in the bundle 903 has been checked in accordance with process 900 (and in some implementations process 900 and process 1100), the bundle or a refined or updated version of the bundle if created, is then sent to the FHIR datastore 122.

In this regard, a FHIR bundle (e.g., FHIR resource bundle 903) has multiple entries, and every entry corresponds to a FHIR resource. In association with receiving clinical data 102, the clinical data, regardless of the initial format in which it is received (e.g., HL7, FHIR or a proprietary JSON or CSV format or other kind of proprietary format), the clinical data is associated with a request action that indicates its purpose or how it should be processed, also called a query type. In this regard, each FHIR resource of the FHIR resource 801 is associated with a query type. In this regard, FHIR resources are designed to work seamlessly with RESTful APIs, enabling CRUD (Create, Read, Update, Delete) operations using standard hypertext transfer protocol (HTTP) methods like GET, POST, PUT, and DELETE operations. In this regard, the GET operations corresponds to a request to read or retrieve a particular FHIR resource, a POST operation corresponds to an explicit request to create a new FHIR resource for the first time, a PUT operation corresponds to a request to update or replace an existing resource or create a new resource if it does not yet exist in the FHIR datastore 122, and a DELETE operation corresponds to a request to delete the FHIR resource in the FHIR datastore 122. Process 900 is particularly concerned with PUT requests, as these requests involve updating an existing resource or an implied request to create a new resource if it does not yet exist in the FHIR datastore 122. On the contrary, POST requests as received through pipeline 100, correspond to explicit requests to create a new FHIR resource and process 900 assumes all POST requests are valid and thus duplication of the FHIR resource in the FHIR datastore 122 is not an issue.

In addition, every FHIR resource included in a new bundle being processed by system 800 (e.g., FHIR resource bundle 903) includes one or more healthcare information identifiers, also referred to herein as healthcare identifiers (HIDs), that uniquely identifies the data as assigned by the healthcare information source from which the data was received. For example, as applied to patients, the HID may include a medical record number (MRN) unique to the organization treating the patient. In another example, as applied to medical image data, the HID may include a unique accession number. In addition, a single FHIR resource may include multiple unique HIDs. For example, a patient resource may also be associated with a particular visit or encounter and include corresponding unique HIDs for the visit or encounter. In addition, every FHIR resource stored in the FHIR datastore 122 includes a unique logical ID (LID), which is the ID used to identify the resource in the FHIR datastore 122.

Sub-process 906 is described in association with processing a single FHIR resource included in FHIR resource bundle 903, identified in FIG. 9 as FHIR resource 903k. The number k of FHIR resources included in the FHIR resource bundle 903 can vary and include one or more resources. As noted above, the data refinement component 118 performs sub-process 906 for each resource in the FHIR resource bundle 903 separately.

In accordance with sub-process 906, at 908, the extraction component first extracts the one or more HIDs for the FHIR resource 903k. At 910, the extraction component identifies or extracts the query type associated with the FHIR resource 903k. As noted above, the query type can include but is not limited to, a GET, POST, PUT, or a DELETE. At 910, the extraction component determines whether the query type is a PUT, that is a request to update the resource or an implied request to create a new resource. If at 910 the extraction component 806 determines that the query request is not a PUT, then it is either a GET, a POST, or a DELETE. In this case, sub-process 906 continues to 914, wherein the identifier resolution component 808 performs no updating of the resource 903k or the bundle 903 related to the resource 903k. However, at 916, the identifier resolution component creates a new entry for the resource 903k in the tracking database 816.

In this regard, tracking database 816 corresponds to a local database or index employed by the data refinement component 118 to track information pertaining to FHIR resources that are already existing in the FHIR datastore 122, referred to herein as existing FHIR resources. In this regard, in association with reviewing a new bundle (e.g., FHIR resource bundle 903) prior to sending the new bundle to the FHIR datastore, process 900 employs tracking database 816 to ensure each resource in the bundle includes the correct LID before sending it to the FHIR datastore 122 and updates the tracking database 816 to include new information for any new resources included in the bundle before sending it to the FHIR datastore 122.

FIG. 10 illustrates an example tracking database 1000, in accordance with one or more embodiments described herein. With reference to FIG. 10 in view of FIGS. 8 and 9, tracking database 1000 corresponds to an example of tracking database 816. As shown in FIG. 10, the tracking database 1000 corresponds to a spreadsheet or index of information pertaining to FHIR resources stored in the FHIR datastore 122. Each row corresponds to a separate FHIR resource. Column A corresponds to the resource type, and identifies the type of the resource (e.g., a patient, a service request, etc.). Column B corresponds to the LID, that is the ID used for the resource in the FHIR datastore 122. In this regard, in addition to the one or more HIDs, each FHIR resource received by system 800 also includes a LID assigned to it that has been generated by the entity or pipeline (e.g., of amongst pipelines 106, 108 and 110) that created it. Each FHIR resource received by the system 800 also includes information indicating the resource type.

Column C corresponds to the one or more HID(s) used for the FHIR resource. Column C corresponds to the sequence number. This is an identifier that tracks the sequence of reception of the resource. In various embodiments, each resource received by the system 800 also includes a sequence number assigned to it indicating the order in which it is received by the system. Additionally, or alternatively, the reception component 804 can assign the sequence numbers to the resources as received, wherein the sequence numbers are numerically ordered in an ascending order such that newer or more recently received resources are given a higher number than older resources. In some implementations, two or more resources may be received at or near the same time. In these scenarios, the sequence number for the respective resources may be the same. For example, as shown in table 1000, sequence number 1001 is applied to both the patient resource in row 2 and service request resource in row 3, indicating reception of those resources at the same time via gateway 104 and/or system 800. The sequence numbers here are thus numerically ordered numbers. However, the sequence numbers may also include or correspond to timestamps.

The source ID (SID) indicates the source from which the resource was received. For example, the source may by an electronic medical record (EMR) system associated with a particular site (e.g., site 1, site 2, etc.), a radiology information system (RIS), a laboratory system, bed management system, a scheduling system, a patient monitoring device or any other type of healthcare information source. Each FHIR resource received by system 800 also includes its source ID.

With reference back to process 900 and sub-process 906, as noted above, if the resource query is not a PUT request, no updating on the resource 903k or bundle 903 related to the resource is needed prior to sending it to the FHIR datastore 122. However, at 916, the identifier resolution component creates a new entry for the resource in the tracking database 816. This corresponds to adding a new row to the tracking database 1000 for the bundle corresponding to the resource 903k. For example, the new entry for the resource will include the information for the resource corresponding to columns A, B, C and D, as extracted from the FHIR resource 903k by the extraction component 806; that is the resource type, the LID, the one or more HIDs, the sequence number and the source ID. At 918, the resource 903k is further maintained in the bundle. Sub-process 906 is further performed for the remaining resources in the bundle 903 until all the resources in the bundle 903 have been reviewed.

If at 910 the extraction component 806 determines that the query request is a PUT request, then at 912, the identifier resolution component 808 checks the tracking database to determine whether the HID(s) for the resource are already included in the tracking database 816. In this regard, if the HID(s) for the resource 903k extracted at 908 are not already referenced in the tracking database 816, this means that the resource 903k does not yet exist in the FHIR datastore 122. Accordingly, if at 912 the identifier resolution component 808 determines that the one or more of the HIDs are not included in the tracking database, process 900 again proceeds to 914, wherein the identifier resolution component 808 performs no updating of the resource 903k or the bundle as related to the resource 903k. However, at 916, the identifier resolution component 808 creates a new entry for the resource 903k in the tracking database 816. At 918, the resource 903k is further maintained in the bundle. Sub-process 906 is further performed for the remaining resources in the bundle 903 until all the resources in the bundle 903 have been reviewed.

If at 912, the identifier resolution component 808 determines that any of the one or more HIDs extracted at 908 are already included in the tracking database 816, this means that the FHIR resource 903k already exists in the FHIR datastore 122. In this case, process 900 proceeds to 920, wherein the extraction component 808 extracts the existing LID corresponding to the existing entry for the resource 903k in the tracking database 816. More particularly, identifier resolution component 808 identifies the entry in the tracking database 816 with the existing HID corresponding to the one or more HIDs extracted at 908 and the same resource type. For example, looking at table 1000, if the FHIR resource is of type Patient and the one or more HIDs extracted at 908 correspond to that shown in the cell corresponding to column C row 2, in that case resource 903k resource is already saved in the FHIR datastore 122 with the existing LID corresponding to column B row 2. In this case, the resource FHIR 903k needs to be updated with its existing LID in the tracking database.

In this regard, at 922, the identifier resolution component 808 updates the FHIR resource 903k to include the existing LID already associated with the resource in the tracking database 816. This corresponds to replacing a new LID associated with the resource 903k as received by system 800 with the existing LID for the resource included in the tracking database 816, converting FHIR resource 903k to updated FHIR resource 903k′. The identifier resolution component 808 also updates the PUT request associated with the resource 903k. What this means is that the identifier resolution component 808 updates the header of the PUT request with the existing LID.

In addition to updating the FHIR resource 903k, at 924 the identifier resolution component 808 updates all (or in some implementations one or more) other references to the same resource in the FHIR bundle 903, with the existing LID. In this regard, as described above with reference to FIG. 2, FHIR resources in a bundle can be related and include reference information referencing related resources. Thus, each time resource 903k is referenced in the bundle, at 924, the identifier resolution component replaces whatever LID is associated with each (or in some implementations one or more) instance of the resource 903k with the existing LID extracted at 920. At 926, the updated FHIR resource 903k′ is then maintained with the bundle 903. Sub-process 906 is further performed for the remaining resources in the bundle 903 until all the resources in the bundle 903 have been reviewed.

In this regard, the result of process 900 includes either the FHIR bundle 903 without any resources updated or an updated or revised version of FHIR bundle 903 with one or more FHIR resources updated with the correct LID, as well as the references to those updated resource being updated FHIR bundle 903 being updated with the correct LID. For example, in some implementations, if the decision is no for each resource following decision blocks 910 and 912, then the FHIR bundle resulting from process 900 corresponds to FHIR resource bundle 903. This means that the FHIR bundle 903 does not include any duplicate instances of a FHIR resource stored in the FHIR datastore 122 with incorrect LIDs. In other implementations in which at least one resource is updated following a yes decision at 912, then the FHIR resource bundle resulting from process 900 corresponds to an updated or refined resource bundle.

In some embodiments, the FHIR resource bundle resulting from process 900 can be sent to the FHIR datastore 122 (by the storage component 814 as refined FHIR resources 803) and stored in the FHIR datastore 122. Additionally, or alternatively, the respective resources included in the FHIR bundle 903 can also be processed in accordance with process 1100 to check and remove resources associated with detected racing conditions.

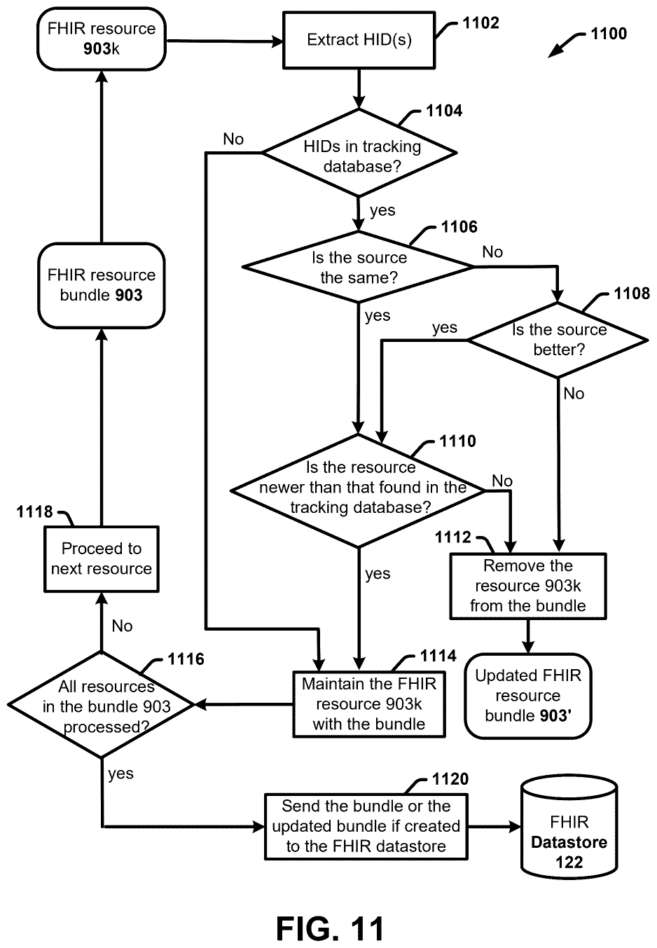

In this regard, FIG. 11 illustrates a high-level flow diagram of an example data refinement process (i.e., process 1100) that facilitates racing condition handling, in accordance with one or more embodiments described herein. With reference to FIG. 11 in view of FIGS. 1, 8, and 9, process 1100 is concerned with racing condition management. In this regard, racing consuming of clinical data in the context of pipeline 100 refers to the ingestion of clinical 102 from multiple healthcare information sources in parallel overtime. Depending on the source and the type of clinical information originating from that source, some clinical data messages may be received via the HL7 to FHIR pipeline 106, via the FHIR pipeline 108 and/or the proprietary to FHIR pipeline 110 and reference the same or a related FHIR resource. In this regard, a racing condition can occur when multiple processes or systems attempt to update or create related FHIR resources simultaneously, leading to inconsistent or unintended states.

For example, let's assume system 800 receives and processes a first FHIR bundle including a new Encounter resource for one specific Patient resource, both resources Encounter and Patient are part of the first bundle received. A second FHIR bundle is received immediately after a few milliseconds with an update of patient demographic information as part of a new version of the first shared Patient resource. If system 800 processes and sends the second bundle to the FHIR datastore 122 before processing and sending the first FHIR bundle to the FHIR datastore 122, it results in overwriting the update to the Patient resource included in the second FHIR bundle with outdated patient information, included in the first FHIR bundle. The result of this racing condition is that the Patient resource in the FHIR datastore 122 would now reflect the state before the update from the second bundle, effectively losing the new demographic information added by the second bundle. In this regard, the Encounter resource has been created successfully, but the integrity of the Patient record is compromised.

In order to prevent or mitigate occurrences of such errors, process 1100 also relies on the information in the tracking database 816, and a rule-based weighting protocol used to determine whether to keep or remove respective resources having exiting entries in the tracking database 816 prior to sending the bundle 903k or a revised version of the bundle 903k′ to the FHIR datastore 122.

Process 1100 involves processing individual FHIR resources included in a FHIR bundle separately (e.g., FHIR resource bundle 903 for example) until all resources have been checked for racing conditions. In this regard, as applied to a FHIR bundle, process 1100 corresponds to an iterative process, wherein at each iteration, a separate FHIR resource included in the bundle (e.g., FHIR resource bundle 903k) is processed before the bundle (or an updated version of the bundle with one or more resources removed) is sent to the FHIR datastore 122. Process 1100 is described for exemplary purposes as applied to a single FHIR resource 903k included in FHIR bundle 903.

In this regard, process 1100 begins at 1102, wherein the extraction component 803 extracts the one or more HIDs from the FHIR resource 903k. At 1104, the racing condition component 812 checks if there is an entry included in the tracking database 816 including any of the one or more of the HIDs. If not, this means that there does not exist a resource in the FHIR datastore 122 related to the same HIDs and thus the resource 903k can be sent to the FHIR datastore 122 without causing a racing condition. In this case, process 1100 proceeds to 1114, wherein the racing condition component 812 maintains the resource 903k with the bundle 903. However, if at 1104 the racing condition component 812 determines that any of the HIDs are in the tracking database 816, then process 1100 proceeds to 1106.

At 1106, the racing condition component 812 then checks the source of the resource 903k to determine whether the source is the same as the existing source for the entry of the resource in the tracking database. In particular, the racing condition component 812 identifies the existing source of the existing resource in the tracking database 816 (e.g., in column E of example tracking database 1000 for example) corresponding to entry with the existing HID corresponding the HID(s) extracted at 1102. For example, as shown in example tracking database 1000, the racing condition component 812 checks the source ID of the existing resource corresponding to the HID(s) and compares it to the source ID associated with resource 903k to determine whether the respective source IDs are the same. If the source IDs are not the same, then process 1100 proceeds to 1108.

At 1108, the racing condition component 812 further determines which of the two resource IDs is better, which corresponds to determining which of the two sources has a stronger or higher weight. In this regard, all of the resources from which clinical information 102 can be received can have predefined weights in accordance with a predefined weighting scheme. Information identifying respective weights of the possible resource IDs can be stored in memory 818. For example, an EMR may have a higher weight than a patient monitoring device. In another example, one radiology information system (RIS_1) may have a higher weight than another RIS (RIS-2). At 1108, if the racing condition component 812 determines that the source ID for resource 903k is not better (or has a lower weight) relative to the existing source ID in the tracking database 816, then process 1100 proceeds to 1112, and the racing condition component 812 removes the resource 903k from the bundle 903, resulting in generation of an updated version of the bundle with the resource 903k removed (e.g., updated FHIR resource bundle 903′).

However, if at 1106, the racing condition component 812 determines that respective source IDs are the same, process 1100 proceeds to 1110. In addition, if at 1108 the racing condition component determines that the new source ID associated with resource 903k is better than the corresponding existing source ID in the tracking database 816, process 1100 also proceeds to 1110. is the same or has a higher weight than the existing resource ID, then process 1100.

At 1110, the racing condition component 812 checks the sequence number associated with resource 903k and compares it to the corresponding existing sequence number for the existing resource in the tracking database 816 (e.g., column D of example tracking database 1000). The racing condition component 812 then determines whether the sequence ID associated with the resource 903k is newer (or higher in number, indicating that it is newer or received by system 800 after the existing resource) than the existing sequence ID for the existing resource. For example, if the sequence number associated with resource 903k is 1001 and the sequence number for the existing resource is lower than 1001 for (e.g., 1000, 999, 998 and so on), then resource 903k is considered newer than the existing resource. In this case, the racing condition component 812 determines that resource 903k is newer than the existing resource, and process 1100 proceeds to 1114, wherein the racing condition component 812 maintains resource 903k with the bundle (FHIR resource bundle 903). However, if the sequence number for resource 903k indicates that it is older than the corresponding existing resource, then process 1100 proceeds to 1112 and the racing condition component 812 removes resource 903k from the bundle, resulting in updated FHIR resource bundle 903′.

As noted above, process 1100 is repeated for each resource in the bundle 903. In this regard, at 1114, the racing condition component 812 determines whether all resources in the FHIR resource bundle 903 have been processed through steps 1102-1114. If not, then process steps 1102-1114 are performed again for the next resource in the bundle, as indicated at 1118. After all the resources in the bundle 903 have been processed through step 1102-1114, the result is either the FHIR bundle 903 as unmodified, or an updated version of the bundle (e.g., updated FHIR resource bundle 903′) with one or more resources removed. At 1120 the storage component 814 sends the resulting bundle to the FHIR datastore 122 and the respective resources in the bundle are then stored in the FHIR datastore 122.

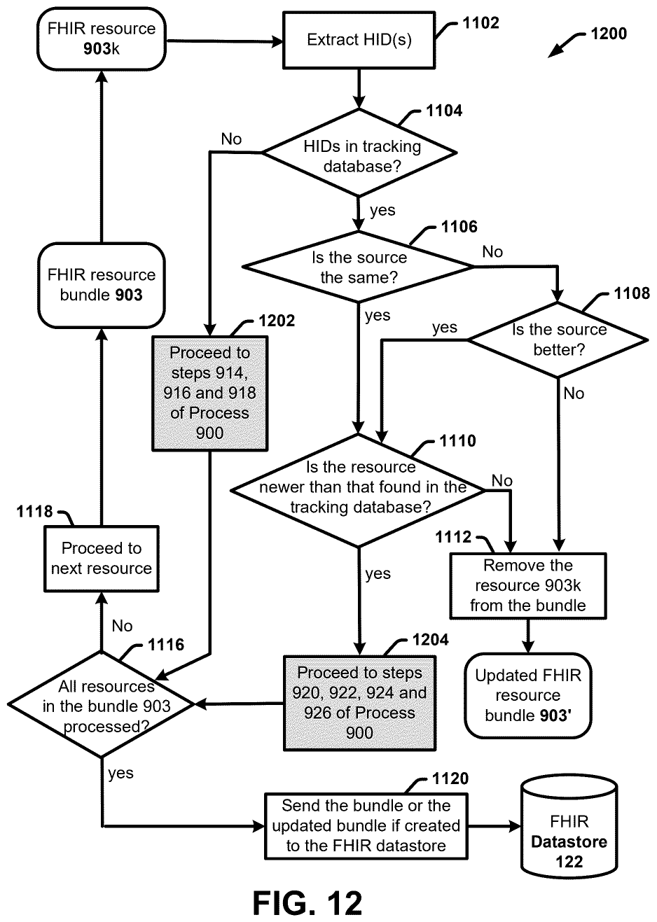

In various embodiments, the data refinement component 118 can perform a combination of process 900 and 1100 as shown in FIG. 12.

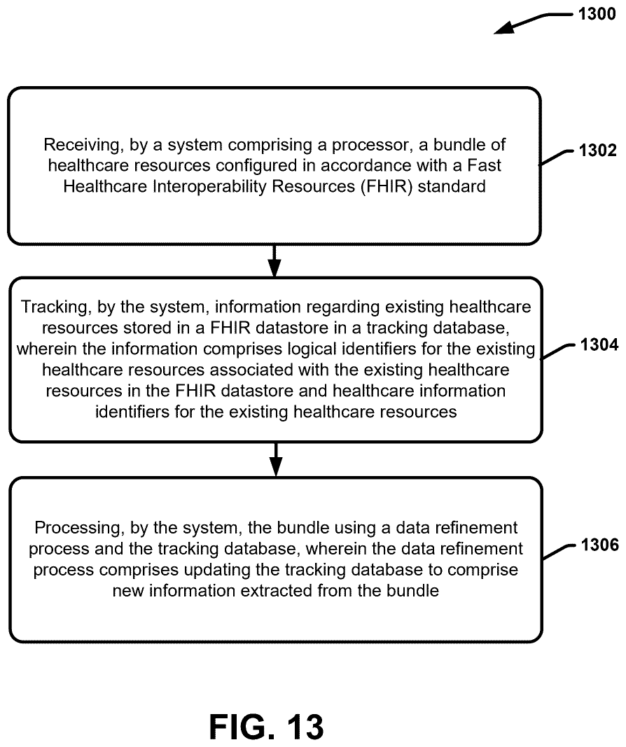

In this regard, FIG. 12 presents another example data refinement process 1200 in accordance with one or more embodiments described herein. With reference to FIG. 13 in view of FIGS. 8-12, process 1200 combines elements of process 900 and process 1100. Process 1200 corresponds to process 1100 with the differences noted at 1202 and 1204. In this regard, in accordance with process 1200, if at 1104 the identifier resolution component 808 determines that the HIDs are not in the tracking database, then process 1200 proceeds to 1202, wherein steps 914, 916 and 918 are performed. In addition, if at 1110 the resource 903k is determined to be newer, then process 1200 proceeds to 1204, wherein steps 920, 922, 924 and 926 are performed. Repetitive description of like elements employed in respective embodiments is omitted for sake of brevity.

FIG. 13 illustrates an example computer-implemented method 1300 that facilitates data deduplication management and clinical data identity resolution, in accordance with one or more embodiments described herein. Method 1300 comprises, at 1302, receiving, by a system comprising a processor (e.g., system 800), a bundle of healthcare resources configured in accordance with a Fast Healthcare Interoperability Resources (FHIR) standard (e.g., via reception component 804). At 1304, method 1300 comprises tracking, by the system (e.g., via tracking database 816 and identifier resolution component 808), information regarding existing healthcare resources stored in a FHIR datastore in a tracking database (e.g., tracking database 816), wherein the information comprises logical identifiers for the existing healthcare resources associated with the existing healthcare resources in the FHIR datastore and healthcare identifiers for the existing healthcare resources. At 1306, method 1300 comprises processing, by the system, the bundle using a data refinement process and the tracking database (e.g., process 900), wherein the data refinement process comprises updating the tracking database to comprise new information extracted from the bundle.

In some embodiments, method 1300 can further comprise sending, by the system (e.g., via storage component 814) the bundle or a refined version of the bundle to the FHIR datastore following the data refinement process.

In some embodiments of method 1300, the data refinement process comprises determining, by the system, whether each resource of the healthcare resources corresponds to an existing healthcare resource stored in the FHIR datastore based on comparison of extracted healthcare identifiers for each resource to the healthcare identifiers for the existing healthcare resources in the tracking database. In some implementations of these embodiments, based on a first determination that a resource of the healthcare resources does not correspond to an existing healthcare resource, the data refinement process further comprises maintaining the resource with the bundle, and updating, by the system, the tracking database to comprise a new entry for the resource.

In other implementations of these embodiments, based on a second determination that a resource of the healthcare resources corresponds to an existing healthcare resource, the data refinement process comprises extracting, by the system, an existing logical identifier for the resource from the tracking database, replacing a new logical identifier associated with the resource with the logical identifier, resulting in an updated version of the resource, and maintaining, by the system, the updated version of the resource with the bundle, resulting in generation of a refined version of the bundle. In some embodiments, the storage component 814 can further sends the refined version of the bundle to the FHIR datastore following the data refinement process. In addition, the identifier resolution component can update all references to the resource in the bundle with the existing logical identifier.

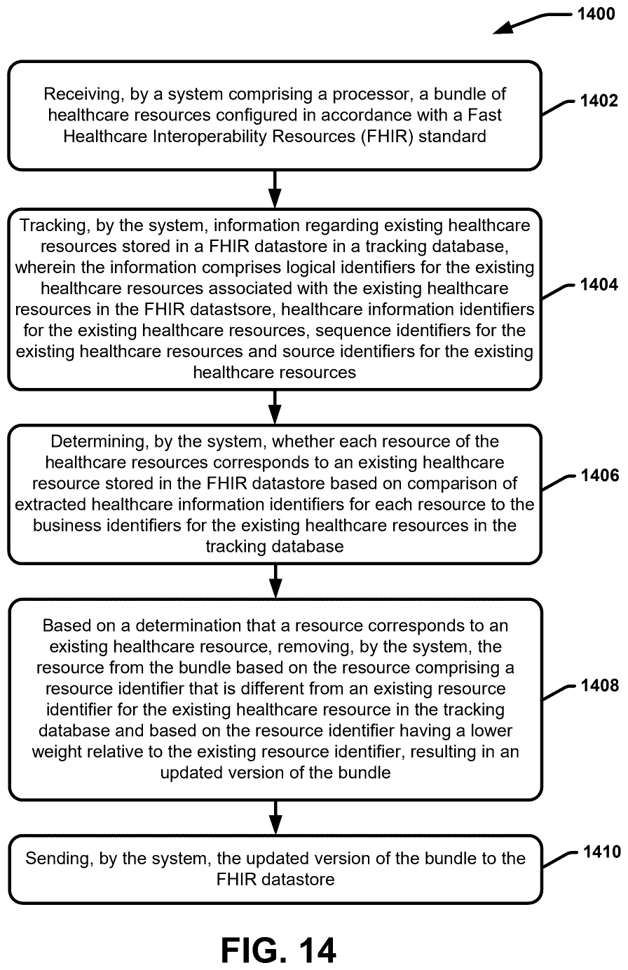

FIG. 14 illustrates an example computer-implemented method 1400 that facilitates racing condition handling, in accordance with one or more embodiments described herein. Method 1400 comprises, at 1402, receiving, by a system comprising a processor (e.g., system 800), a bundle of healthcare resources configured in accordance with a Fast Healthcare Interoperability Resources (FHIR) standard (e.g., via reception component 804). At 1404, method 1400 comprises tracking, by the system (e.g., via tracking database 816 and identifier resolution component 808), information regarding existing healthcare resources stored in a FHIR datastore in a tracking database (e.g., tracking database 816), wherein the information comprises logical identifiers for the existing healthcare resources associated with the existing healthcare resources in the FHIR datastore, healthcare identifiers for the existing healthcare resources, sequence identifiers for the existing healthcare resources and source identifiers for the existing healthcare resources. At 1406, method 1400 comprises determining, by the system, whether each resource of the healthcare resources corresponds to an existing healthcare resource stored in the FHIR datastore based on comparison of extracted healthcare identifiers for each resource to the healthcare identifiers for the existing healthcare resources in the tracking database. At 1408, method 1400 comprises, based on a determination that a resource corresponds to an existing healthcare resource, removing, by the system, the resource from the bundle based on the resource comprising a resource identifier that is different from an existing resource identifier for the existing healthcare resource in the tracking database and based on the resource identifier having a lower weight relative to the existing resource identifier, resulting in an updated version of the bundle. At 1410, method 1400 comprises sending, by the system, the updated version of the bundle to the FHIR datastore.

In some embodiments, based on another determination that a resource corresponds to an existing healthcare resource, method 1400 can further comprise removing the resource in the bundle based on the resource comprising a resource identifier that is different from an existing resource identifier for the existing healthcare resource in the tracking database, and based on the resource identifier having a higher eight relative to the existing resource identifier, yet the resource is older (based on the respective sequence identifiers) than the existing resource.

In some embodiments, based on another determination that a resource corresponds to an existing healthcare resource, method 1400 can further comprise maintaining the resource in the bundle based on the resource comprising a resource identifier that is different from an existing resource identifier for the existing healthcare resource in the tracking database, yet the resource has a source ID that has a higher weight than the corresponding existing source ID and the resource is newer than the existing resource.

Still in other embodiments, based on another determination that a resource corresponds to an existing healthcare resource, method 1400 can further comprise maintaining the resource in the bundle based on the resource comprising a resource identifier that is the same as an existing resource identifier for the existing healthcare resource in the tracking database, and wherein the resource is newer than the existing resource.

Method 1400 can further comprise maintaining the resource with the bundle based on another determination that the resource does not correspond to an existing healthcare resource.

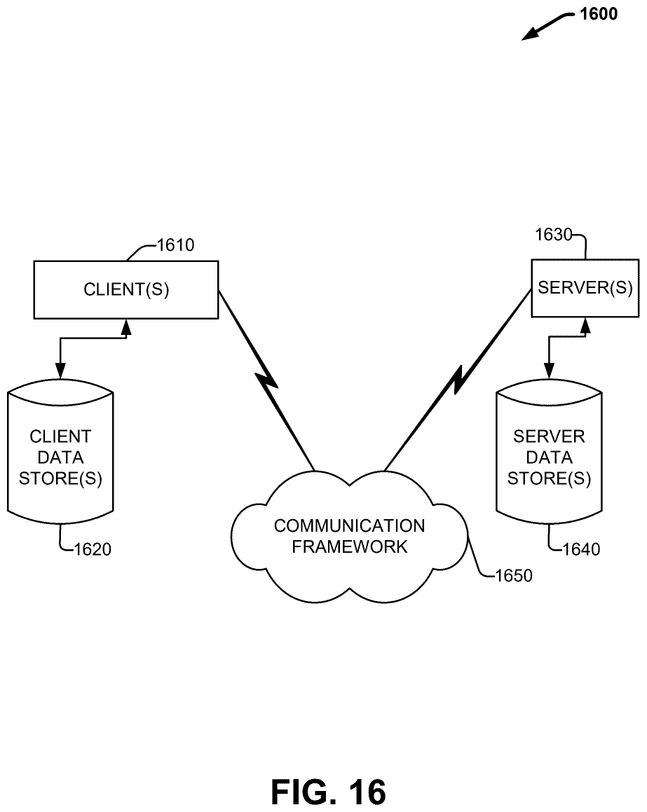

In order to provide a context for the various aspects of the disclosed subject matter, FIGS. 15 and 16 as well as the following discussion are intended to provide a brief, general description of a suitable environment in which the various aspects of the disclosed subject matter may be implemented.

With reference to FIG. 15, a suitable environment 1500 for implementing various aspects of this disclosure includes a computer 1512. The computer 1512 includes a processing unit 1514, a system memory 1516, and a system bus 1518. The system bus 1518 couples system components including, but not limited to, the system memory 1516 to the processing unit 1514. The processing unit 1514 can be any of various available processors. Dual microprocessors and other multiprocessor architectures also can be employed as the processing unit 1514.

The system bus 1518 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1516 includes volatile memory 1520 and nonvolatile memory 1522. The basic input/output system (BIOS), containing the basic routines to transfer information between elements within the computer 1512, such as during start-up, is stored in nonvolatile memory 1522. By way of illustration, and not limitation, nonvolatile memory 1522 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 1520 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 1512 also includes removable/non-removable, volatile/non-volatile computer storage media. FIG. 15 illustrates, for example, a disk storage 1524. Disk storage 1524 includes, but is not limited to, devices like a magnetic disk drive, floppy disk drive, tape drive, Jaz drive, Zip drive, LS-150 drive, flash memory card, or memory stick. The disk storage 1524 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1524 to the system bus 1518, a removable or non-removable interface is typically used, such as interface 1526.

FIG. 15 also depicts software that acts as an intermediary between users and the basic computer resources described in the suitable operating environment 1500. Such software includes, for example, an operating system 1528. Operating system 1528, which can be stored on disk storage 1524, acts to control and allocate resources of the computer system 1512. System applications 1530 take advantage of the management of resources by operating system 1528 through program modules 1532 and program data 1534, e.g., stored either in system memory 1516 or on disk storage 1524. It is to be appreciated that this disclosure can be implemented with various operating systems or combinations of operating systems.

A user enters commands or information into the computer 1512 through input device(s) 1536. Input devices 1536 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1514 through the system bus 1518 via interface port(s) 1538. Interface port(s) 1538 include, for example, a serial port, a parallel port, a game port, and a universal serial bus (USB). Output device(s) 1540 use some of the same type of ports as input device(s) 1536. Thus, for example, a USB port may be used to provide input to computer 1512, and to output information from computer 1512 to an output device 1540. Output adapter 1542 is provided to illustrate that there are some output devices 1540 like monitors, speakers, and printers, among other output devices 1540, which require special adapters. The output adapters 1542 include, by way of illustration and not limitation, video and sound cards that provide a means of connection between the output device 1540 and the system bus 1518. It should be noted that other devices and/or systems of devices provide both input and output capabilities such as remote computer(s) 1544.

Computer 1512 can operate in a networked environment using logical connections to one or more remote computers, such as remote computer(s) 1544. The remote computer(s) 1544 can be a personal computer, a server, a router, a network PC, a workstation, a microprocessor based appliance, a peer device or other common network node and the like, and typically includes many or all of the elements described relative to computer 1512. For purposes of brevity, only a memory storage device 1546 is illustrated with remote computer(s) 1544. Remote computer(s) 1544 is logically connected to computer 1512 through a network interface 1548 and then physically connected via communication connection 1550. Network interface 1548 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1550 refers to the hardware/software employed to connect the network interface 1548 to the bus 1518. While communication connection 1550 is shown for illustrative clarity inside computer 1512, it can also be external to computer 1512. The hardware/software necessary for connection to the network interface 1548 includes, for exemplary purposes only, internal and external technologies such as, modems including regular telephone grade modems, cable modems and DSL modems, ISDN adapters, and Ethernet cards.

FIG. 16 is a schematic block diagram of a sample-computing environment 1600 with which the subject matter of this disclosure can interact. The system 1600 includes one or more client(s) 1610. The client(s) 1610 can be hardware and/or software (e.g., threads, processes, computing devices). The system 1600 also includes one or more server(s) 1630. Thus, system 1600 can correspond to a two-tier client server model or a multi-tier model (e.g., client, middle tier server, data server), amongst other models. The server(s) 1630 can also be hardware and/or software (e.g., threads, processes, computing devices). The servers 1630 can house threads to perform transformations by employing this disclosure, for example. One possible communication between a client 1610 and a server 1630 may be in the form of a data packet transmitted between two or more computer processes.

The system 1600 includes a communication framework 1650 that can be employed to facilitate communications between the client(s) 1610 and the server(s) 1630. The client(s) 1610 are operatively connected to one or more client data store(s) 1620 that can be employed to store information local to the client(s) 1610. Similarly, the server(s) 1630 are operatively connected to one or more server data store(s) 1640 that can be employed to store information local to the servers 1630.

It is to be noted that aspects or features of this disclosure can be exploited in substantially any wireless telecommunication or radio technology, e.g., Wi-Fi; Bluetooth; Worldwide Interoperability for Microwave Access (WiMAX); Enhanced General Packet Radio Service (Enhanced GPRS); Third Generation Partnership Project (3GPP) Long Term Evolution (LTE); Third Generation Partnership Project 2 (3GPP2) Ultra Mobile Broadband (UMB); 3GPP Universal Mobile Telecommunication System (UMTS); High Speed Packet Access (HSPA); High Speed Downlink Packet Access (HSDPA); High Speed Uplink Packet Access (HSUPA); GSM (Global System for Mobile Communications) EDGE (Enhanced Data Rates for GSM Evolution) Radio Access Network (GERAN); UMTS Terrestrial Radio Access Network (UTRAN); LTE Advanced (LTE-A); etc. Additionally, some or all of the aspects described herein can be exploited in legacy telecommunication technologies, e.g., GSM. In addition, mobile as well non-mobile networks (e.g., the Internet, data service network such as internet protocol television (IPTV), etc.) can exploit aspects or features described herein.

While the subject matter has been described above in the general context of computer-executable instructions of a computer program that runs on a computer and/or computers, those skilled in the art will recognize that this disclosure also can or may be implemented in combination with other program modules. Generally, program modules include routines, programs, components, data structures, etc. that perform particular tasks and/or implement particular abstract data types. Moreover, those skilled in the art will appreciate that the inventive methods may be practiced with other computer system configurations, including single-processor or multiprocessor computer systems, mini-computing devices, mainframe computers, as well as personal computers, hand-held computing devices (e.g., PDA, phone), microprocessor-based or programmable consumer or industrial electronics, and the like. The illustrated aspects may also be practiced in distributed computing environments where tasks are performed by remote processing devices that are linked through a communications network. However, some, if not all aspects of this disclosure can be practiced on stand-alone computers. In a distributed computing environment, program modules may be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

In another example, respective components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

Various aspects or features described herein can be implemented as a method, apparatus, system, or article of manufacture using standard programming or engineering techniques. In addition, various aspects or features disclosed in this disclosure can be realized through program modules that implement at least one or more of the methods disclosed herein, the program modules being stored in a memory and executed by at least a processor. Other combinations of hardware and software or hardware and firmware can enable or implement aspects described herein, including a disclosed method(s). The term “article of manufacture” as used herein can encompass a computer program accessible from any computer-readable device, carrier, or storage media. For example, computer readable storage media can include but are not limited to magnetic storage devices (e.g., hard disk, floppy disk, magnetic strips . . . ), optical discs (e.g., compact disc (CD), digital versatile disc (DVD), blu-ray disc (BD) . . . ), smart cards, and flash memory devices (e.g., card, stick, key drive . . . ), or the like.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor may also be implemented as a combination of computing processing units.

In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory.

By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or methods herein are intended to include, without being limited to including, these and any other suitable types of memory.

It is to be appreciated and understood that components, as described with regard to a particular system or method, can include the same or similar functionality as respective components (e.g., respectively named components or similarly named components) as described with regard to other systems or methods disclosed herein.

What has been described above includes examples of systems and methods that provide advantages of this disclosure. It is, of course, not possible to describe every conceivable combination of components or methods for purposes of describing this disclosure, but one of ordinary skill in the art may recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

What is claimed is:

1. A system, comprising:
at least one memory that stores computer-executable components; and
at least one processor that executes the computer-executable components stored in the at least one memory, wherein the computer-executable components comprise:
a reception component that receives a bundle of healthcare resources configured in accordance with a Fast Healthcare Interoperability Resources (FHIR) standard;
a tracking database that tracks information regarding existing healthcare resources stored in a FHIR datastore, wherein the information comprises logical identifiers for the existing healthcare resources associated with the existing healthcare resources in the FHIR datastore and healthcare identifiers for the existing healthcare resources; and
a data refinement component that processes the bundle using a data refinement process and the tracking database and updates the tracking database to comprise new information extracted from the bundle.

2. The system of claim 1, wherein the computer-executable components further comprise:
a storage component that sends the bundle or a refined version of the bundle to the FHIR datastore following the data refinement process.

3. The system of claim 1, wherein the data refinement process comprises:
determining whether each resource of the healthcare resources corresponds to an existing healthcare resource stored in the FHIR datastore based on comparison of extracted healthcare identifiers for each resource to the healthcare identifiers for the existing healthcare resources in the tracking database.

4. The system of claim 3, wherein based on a determination that a resource of the healthcare resources does not correspond to an existing healthcare resource, the data refinement process comprises:
maintaining the resource with the bundle; and
updating the tracking database to comprise a new entry for the resource.

5. The system of claim 3, wherein based on a determination that a resource of the healthcare resources corresponds to an existing healthcare resource, the data refinement process comprises:
extracting an existing logical identifier for the resource from the tracking database;
replacing a new logical identifier associated with the resource with the logical identifier, resulting in an updated version of the resource; and
maintaining the updated version of the resource with the bundle, resulting in generation of a refined version of the bundle.

6. The system of claim 5, wherein the computer-executable components further comprise:
a storage component that sends the refined version of the bundle to the FHIR datastore following the data refinement process.

7. The system of claim 5, wherein based on the determination that the resource of the corresponds to the existing healthcare resource, the data refinement process further comprises:
updating one or more other references to the resource in the bundle with the existing logical identifier.

8. The system of claim 7, wherein the information in the tracking database further comprises sequence identifiers for the existing healthcare resources and source identifiers for the existing healthcare resources, and wherein based on a determination that a resource of the healthcare resources corresponds to an existing healthcare resource, the data refinement process comprises:
determining whether a source identifier of the source identifiers corresponding to the existing healthcare resource corresponds to a new source identifier associated with the resource;
based on a second determination that the source identifier does not correspond to the new source identifier; and
determining whether the new source identifier has a higher weight than the source identifier.

9. The system of claim 8, wherein the data refinement process further comprises:
based on a third determination that the new source identifier does not have a higher weight than the source identifier, removing the resource from the bundle, resulting in generation of a refined version of the bundle.

10. The system of claim 8, wherein the data refinement process further comprises:
based on a third determination that the new source identifier has a higher weight than the source identifier, determining whether a new sequence identifier associated with the resource is higher than a sequence identifier of the sequence identifiers corresponding to the existing healthcare resource in the tracking database.

11. The system of claim 10, wherein based on a fourth determination that the new sequence identifier is not higher, removing the resource from the bundle, resulting in generation of a refined version of the bundle.

12. The system of claim 10, wherein based on a fourth determination that the new sequence identifier is higher, maintaining the resource with the bundle.

13. The system of claim 1, wherein the computer-executable components further comprise:
a proprietary to FHIR pipeline that converts clinical data in a proprietary format into the healthcare resources configured in accordance with the FHIR standard based on mapping data elements extracted from the clinical data to one or more FHIR profiles defined for the clinical data.

14. A method, comprising:
receiving, by a system comprising a processor, a bundle of healthcare resources configured in accordance with a Fast Healthcare Interoperability Resources (FHIR) standard;
tracking, by the system, information regarding existing healthcare resources stored in a FHIR datastore in a tracking database, wherein the information comprises logical identifiers for the existing healthcare resources associated with the existing healthcare resources in the FHIR datastore and healthcare identifiers for the existing healthcare resources; and
processing, by the system, the bundle using a data refinement process and the tracking database, wherein the data refinement process comprises updating the tracking database to comprise new information extracted from the bundle.

15. The method of claim 14, further comprising:
sending, by the system, the bundle, or a refined version of the bundle resulting from the data refinement process, to the FHIR datastore following the data refinement process.

16. The method of claim 14, wherein the data refinement process comprises:
determining, by the system, whether each resource of the healthcare resources corresponds to an existing healthcare resource stored in the FHIR datastore based on comparison of extracted healthcare identifiers for each resource to the healthcare identifiers for the existing healthcare resources in the tracking database.

17. The method of claim 16, wherein based on a determination that a resource of the healthcare resources does not correspond to an existing healthcare resource, the data refinement process comprises:
maintaining, by the system, the resource with the bundle; and
updating, by the system, the tracking database to comprise a new entry for the resource.

18. The method of claim 16, wherein based on a determination that a resource of the healthcare resources corresponds to an existing healthcare resource, the data refinement process comprises:
extracting, by the system, an existing logical identifier for the resource from the tracking database;
replacing, by the system, a new logical identifier associated with the resource with the logical identifier, resulting in an updated version of the resource;
maintaining, by the system, the updated version of the resource with the bundle, resulting in generation of a refined version of the bundle; and
sending, by the system, the refined version of the bundle to the FHIR datastore following the data refinement process.

19. The method of claim 14, wherein the information in the tracking database further comprises sequence identifiers for the existing healthcare resources and source identifiers for the existing healthcare resources, and wherein based on a determination that a resource of the healthcare resources corresponds to an existing healthcare resource, the data refinement process comprises:
removing, by the system, the resource from the bundle based on a second determination that a source identifier of the source identifiers does not correspond to the existing healthcare resource and a third determination that the new source identifier has a lower weight than the source identifier, resulting in generation of a refined version of the bundle; and
sending, by the system, the refined version of the bundle to the FHIR datastore.

20. A non-transitory machine-readable storage medium, comprising executable instructions that, when executed by a processor, facilitate performance of operations, comprising:
receiving a bundle of healthcare resources configured in accordance with a Fast Healthcare Interoperability Resources (FHIR) standard;
tracking information regarding existing healthcare resources stored in a FHIR datastore in a tracking database, wherein the information comprises logical identifiers for the existing healthcare resources associated with the existing healthcare resources in the FHIR datastore and healthcare identifiers for the existing healthcare resources; and
processing the bundle using a data refinement process and the tracking database, wherein the data refinement process comprises updating the tracking database to comprise new information extracted from the bundle.
